(19) United States
(12) Patent Application Publication
(10) Pub. No.: US 2025/0115501 A1
(43) Pub. Date: Apr. 10, 2025

(54) CYNOBACTERIA CULTIVATION, SEDIMENTATION, AND UTILIZATION

(71) Applicant: THE TEXAS A&M UNIVERSITY SYSTEM, College Station, TX (US)

(72) Inventors: Shuhua Yuan, College Station, TX (US); Bin Long, College Station, TX (US)

(21) Appl. No.: 18/728,783

(22) PCT Filed: Jan. 13, 2023
(86) PCT No.: PCT/US2023/010815

§ 371 Date: Jul. 12, 2024

Related U.S. Application Data
(60) Provisional application No. 63/299,162, filed on Jan. 13, 2022.

(51) Int. Cl. C02F 3/34; C02F 101/32; C02F 101/34; C02F 103/00; C12N 1/20; C12N 9/88

(52) U.S. Cl. CPC C02F 3/34; C12N 1/20; C12N 9/88; C12Y 402/03016; C02F 2101/32; C02F 2101/34; C02F 2103/007

(57) ABSTRACT

The present invention is related to the fields of bioplastics. In particular, a strain of photosynthetic cyanobacteria (i.e. blue-green algae) having a high growth rate was engineered to overexpress limonene synthase. These engineered cyanobacteria improve auto-sedimentation for biomass harvest and provide for plastic bioremediation capture. Further, machine-learning methods are provided for improving some of the aspects of cultivating cyanobacteria including, but not limited to, assessing real-time light availability and predicting real-time algae growth rates to increase a cyanobacteria biomass.

### CROSS-REFERENCE TO RELATED APPLICATIONS

This is a national entry application claims priority to PCT/US2023/010815 filed Nov. 13, 2023, which claims priority to Provisional Application Ser. No. 63/299,162 filed Jan. 13, 2022 under U.S.C. § 111(b), which is incorporated herein by reference.

### FIELD OF THE INVENTION

The present invention is related to the fields of bioplastics. In particular, a strain of photosynthetic cyanobacteria (i.e. blue-green algae) having a high growth rate was engineered to overexpress limonene synthase. These engineered cyanobacteria improve auto-sedimentation for biomass harvest and provide for plastic bioremediation capture. Further, machine-learning methods are provided for improving some of the aspects of cultivating cyanobacteria including, but not limited to, assessing real-time light availability and predicting real-time algae growth rates to increase a cyanobacteria biomass.

### BACKGROUND

Algae-based bioproduction represents one of the most energy- and carbon-efficient solutions for renewable fuels and CO₂ capture and utilization. Wang, et al., Photosynthetic terpene hydrocarbon production for fuels and chemicals. Plant Biotechnol J 13, 137-146 (2015). Despite significant potential and extensive efforts, the commercialization of algal biofuel has been hindered by one or more of limited sunlight penetration into growth chambers or growth areas, e.g. ponds, poor cultivation dynamics, relatively low yield, and the absence of cost-effective industrial harvest methods. Singh, et al., Microalgae as second-generation biofuel. A review. Agron Sustain Dev 31, 605-629 (2011); Milledge, et al., A review of the harvesting of micro-algae for biofuel production. Rev Environ Sci Bio 12, 165-178 (2013); Rawat, et al., Biodiesel from microalgae: A critical evaluation from laboratory to large scale production. Appl Energ 103, 444-467 (2013); Barros, et al., Harvesting techniques applied to microalgae: A review. Renew Sust Energ Rev 41, 1489-1500 (2015); Gupta, et al., A mini review: photobioreactors for large scale algal cultivation. World J Microb Biot 31, 1409-1417 (2015).

Growth limitation caused by mutual shading and high dewatering costs are among major causes for these technical barriers. Lam, et al., Microalgae biofuels: A critical review of issues, problems and the way forward. Biotechnol Adv 30, 673-690 (2012); Mata, et al., Microalgae for biodiesel production and other applications: A review. Renew Sust Energ Rev 14, 217-232 (2010); Wang, et al., The difference in effective light penetration may explain the superiority in photosynthetic efficiency of attached cultivation over the conventional open pond for microalgae. Biotechnol Biofuels 8, (2015).

Overcoming these challenges would enable viable algal biofuels to provide benefits over current types of production, such as reducing carbon emissions, mitigating climate change, alleviating petroleum dependency, and transforming the bioeconomy.

### SUMMARY OF THE INVENTION

The present invention is related to the fields of bioplastics. In particular, a strain of photosynthetic cyanobacteria (i.e. blue-green algae) having a high growth rate was engineered to overexpress limonene synthase. These engineered cyanobacteria improve auto-sedimentation for biomass harvest and provide for plastic bioremediation capture. Further, machine-learning methods are provided for improving some of the aspects of cultivating cyanobacteria including, but not limited to, assessing real-time light availability and predicting real-time algae growth rates to increase a cyanobacteria biomass.

In one embodiment, the present invention contemplates a method, comprising: a) providing; i) a plurality of photosynthetic cyanobacterial cells comprising a transgene comprising a codon optimized plant limonene synthase gene; and ii) a body of water comprising a plurality of plastic particles; b) overexpressing the transgene to display at least one limonene moiety on the surface of the photosynthetic cyanobacterial cells; c) capturing a portion of the plurality of plastic particles with the displayed at least one limonene moiety to create a limonene-plastic particle complex; and c) sedimenting said limonene-plastic particle complex. In one embodiment, the photosynthetic cyanobacterial cells are Synechococcus (S.) elongatus UTEX 2973 cells. In one embodiment, the body of water is a body of wastewater. In one embodiment, the body of water is a naturally occurring body of water. In one embodiment, the naturally occurring body of water comprises fresh water. In one embodiment, the naturally occurring body of water comprises salt water. In one embodiment, the plurality of plastic particles comprise at least one polymer that includes, but is not limited to, polyethylene, polyethylene terephthalate, polystyrene and carboxymethyl cellulose. In one embodiment, the limonene-plastic particle complex is a bioplastic composite. In one embodiment, the plurality of plastic particles are a plurality of plastic microparticles. In one embodiment, the plurality of plastic particles are a plurality of plastic nanoparticles. In one embodiment, the capturing comprises a hydrophobic interaction between the plurality of plastic particles and the displayed at least one limonene moiety. In one embodiment, the codon optimized limonene synthase gene is a plant limonene synthase gene. In one embodiment, the plant limonene synthase gene is a mint limonene synthase gene. In one embodiment, the mint limonene synthase gene is a Mentha spicata limonene synthase gene. In one embodiment, said transgene is SEQ ID NO. 1.

In one embodiment, the present invention contemplates a composition comprising a photosynthetic cyanobacteria comprising a transgene encoding a codon optimized limonene synthase gene. In one embodiment, the photosynthetic cyanobacteria overexpresses a limonene synthase protein. In one embodiment, the codon optimized limonene synthase gene is a plant limonene synthase gene. In one embodiment, the plant limonene synthase gene is a mint limonene synthase gene. In one embodiment, the mint limonene synthase gene is a Mentha spicata limonene synthase gene. In one embodiment, the photosynthetic cyanobacteria is Synechococcus (S.) elongatus. In one embodiment, the S. elongatus is a UTEX 2973 strain. In one embodiment, said overexpressed limonene synthase protein displays a limonene moiety on the surface of said S. elongatus. In one embodiment, the transgene is operably linked to a constitutive promoter. In one embodiment, said transgene is SEQ ID NO. 1. In one embodiment, the composition further comprises a liquid. In one embodiment, said liquid comprises a plurality of plastic nanoparticles. In one embodiment, the plurality of plastic nanoparticles in the liquid comprise a concentration selected from the group consisting of 0.001%, 0.005%, 0.01%, 0.02%, and 0.05%, or up to 0.05% (w/v). In one embodiment, the plurality of plastic nanoparticles comprise at least one polymer including, but not limited to polyethylene, polyethylene terephthalate, polystyrene and carboxymethyl cellulose. In one embodiment, the plurality of plastic nanoparticles comprise an average diameter of approximately 200, 400 or 800 nm. In one embodiment, the plurality of plastic nanoparticles comprise an average diameter of approximately 1, 2 or 5 micrometers.

In one embodiment, the present invention contemplates a method, comprising: a) providing; i) a plurality of photosynthetic cyanobacteria cells; ii) a body of water; iii) an agitation device placed within the body of water; and iv) a light source emitting light onto the body of water; b) mixing the plurality of photosynthetic cyanobacteria cells into the body of water; c) cultivating the plurality of photosynthetic cyanobacteria cells in the presence of the emitted light; and d) adjusting the intensity of the emitted light or removing a portion of the cultivated plurality of photosynthetic cyanobacteria cells to maintain a predicted growth rate of the cultivated plurality of photosynthetic cyanobacteria cells. In one embodiment, the method further comprises circulating the plurality of photosynthetic cyanobacteria cells with the agitation device. In one embodiment, the adjusting is predicted by machine learning. In one embodiment, the photosynthetic cyanobacteria cell is an S. elongatus UTEX 2973 cell comprising a codon optimized limonene synthase gene. In one embodiment, the body of water includes, but is not limited to, a pool, pond, bioreactor, collection container and/or a centrifuge tube. In one embodiment, said removing the portion of the plurality of photosynthetic cyanobacteria cells modulates a growth parameter including, but not limited to, reduced mutual shading levels, increased light intensity levels and/or increased growth rate. In one embodiment, said modulated growth parameter increases cyanobacterial biomass productivity. In one embodiment, the adjusting of the light intensity is controlled with a turbidity meter. In one embodiment, the adjusting of the light intensity is controlled with a light sensor. In one embodiment, the removing of the portion of the plurality of cyanobacteria cells results in a cyanobacteria cell concentration of approximately 2.3 OD₇₃₀.

In one embodiment, the present invention contemplates a composition comprising a bioplastic composite, the composite comprising a plurality of bioremediated plastic nanoparticles and at least one photosynthetic cyanobacteria polymer. In one embodiment, the bioplastic composite is a film. In one embodiment, the photosynthetic cyanobacteria polymer is derived from a Synechococcus elongatus UTEX 2973 strain. In one embodiment, the Synechococcus elongatus UTEX 2973 strain comprises a codon optimized limonene synthase transgene.

In one embodiment, the present invention contemplates a method, comprising: a) providing, i) a plurality of Synechococcus elongatus UTEX 2973 cells comprising a codon optimized limonene synthase transgene, ii) a plurality of plastic nanoparticles; b) contacting said plurality of Synechococcus elongatus UTEX 2973 cells with said plurality of plastic nanoparticles; and c) co-sedimenting the plurality of Synechococcus elongatus UTEX 2973 cells and the plurality of plastic nanoparticles to create a bioplastic composite. In one embodiment, the bioplastic composite is a film. In one embodiment, the plurality of Synechococcus elongatus UTEX 2973 cells are L524 cells. In one embodiment, the method further comprises settling the plurality of Synechococcus elongatus UTEX 2973 cells for approximately one hour. In one embodiment, the method further comprises centrifuging approximately five (5) milliliters of the bioplastic composite at 7,000 rpm for 10 minutes. In one embodiment, the method further comprises lyophilizing the approximately five (5) milliliters of the bioplastic composite prior to centrifugation. In one embodiment, the method further comprises adding three (3) ml of a chloromethane or a trihalomethane to the approximately five (5) milliliters of the bioplastic composite prior to lyophilization. In one embodiment, the method further comprises sonicating the lyophilized bioplastic composite for ten (10) seconds-on and one hundred (100) seconds-off for at least six (6) cycles. In one embodiment, the method further comprises centrifuging the sonicated bioplastic composite at ten thousand (10,000) rpm for 10 mins. In one embodiment, the method further comprises filtering a casing film from a supernatant. In one embodiment, said bioplastic nanoparticles comprise polystyrene. In one embodiment, the plurality of Synechococcus elongatus UTEX 2973 cells have a concentration determined by an optical density(730) of fifteen (15). In one embodiment, said bioplastic nanoparticles are in a solution having a concentration of approximately 0.1 g/L.

In one embodiment, the present invention contemplates a method, comprising: a) providing, i) a plurality of Synechococcus (S.) elongatus UTEX 2973 strain of photosynthetic cyanobacteria engineered for expressing a transgene comprising a codon optimized limonene synthase, ii) a body of water, ii) a carbohydrate-based nanoparticle, and b) introducing said engineered S. elongatus UTEX 2973 strain and said nanoparticle in said body of water, wherein said engineered S. elongatus UTEX 2973 strain auto-sediments with said nanoparticle. In one embodiment, wherein said nanoparticle is carboxy-methyl cellulose. In one embodiment, said sediment is cast into a bioplastic, e.g., a carbohydrate-based film.

In one embodiment, the present invention contemplates a method comprising: a) providing; i) a S. elongatus UTEX 2973 strain, and ii) a transgene comprising a codon optimized plant limonene synthase gene, b) transforming said S. elongatus UTEX 2973 strain with said transgene; and c) overexpressing a plant limonene synthase gene. In one embodiment, the transgene is operably linked to a constitutive promoter. In one embodiment, said S. elongatus UTEX 2973 displays a limonene moiety on a cell surface. In one embodiment, the limonene moiety increases the hydrophobicity of the cell surface.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” or “approximately” as used herein, in the context of any of any assay measurements refers to +/−5% of a given measurement.

As used herein, “bioplastic composite” refers to a composition comprising bioremediated plastic particles bound to at least one polymer derived from a cyanobacteria.

As used herein, “plastic film” or “bioplastic film” refers to a continuous polymeric material. Thicker plastic film is often referred to as a “sheet”.

As used herein, “bioplastic” refers to a type of biodegradable plastic derived from biological substances rather than from petroleum.

As used herein, “biofilm” refers to a community of bacteria enclosed in a self-produced exopolysaccharide matrix that adheres to a biotic or abiotic surface.

As used herein, “cyanobacteria” and “blue-green algae” refer to a group of photosynthetic bacteria widely distributed throughout aquatic environments, including freshwater and marine (saltwater) environments, and in some soils. Cyanobacteria may occur as single cells, thread-like filaments, or as colonies of various sizes and shapes composed of groups of many filaments or cells.

As used herein, “photosynthesis” refers to a process including chlorophyl, pigments, and chromophores for converting light energy into chemical energy. Some of this chemical energy is stored in carbohydrate molecules, such as sugars and starches, using sunlight to synthesize foods from carbon dioxide and water, also producing oxygen. Oxygen is released as a waste product. chlorophyl, pigments, and chromophores

As used herein, “photosynthetic pigments” refer to molecules that absorb light and have a color including chlorophylls, carotenoids, and phycobilins.

As used herein, “PCC 7942” and “Synechococcus elongatus PCC 7942” refers to a strain of cyanobacteria.

As used herein, “UTEX 2973” and “Synechococcus elongatus UTEX 2973 “refers to a strain of cyanobacteria.

As used herein, “L524” refers to a Synechococcus elongatus UTEX 2973 cyanobacteria strain engineered with a transgenic construct resulting in overexpressing limonene synthase for producing limonene.

As used herein, “strain” refers to a plurality of cyanobacteria cells.

As used herein, “limonene” refers to a liquid hydrocarbon, e.g. olefin hydrocarbon (C₁₀H₁₆).

The term “synonymous codon”, as used herein, refers to different codons that encode the same amino acid. This phenomenon is often referred to as “degeneracy” of the genetic code.

For example, six different codons encode the amino acid arginine.

As used herein, “codon optimized” refers to the use of synonymous codon changes that result in mRNA transcription overexpression from a target gene with a concomitant increases in protein expression. Applications for codon-optimization include recombinant protein drugs and nucleic acid therapies, including gene therapy, mRNA therapy, and DNA/RNA vaccines. For example, “a codon optimized” target gene overexpresses an encoded protein.

As used herein, “agitate” refers to keeping particles suspended in a body of liquid, rather than let these particles settle towards the lower surface of a body of liquid. Agitate also refers to any of chum, stir, swirl, whirl, and the like.

As used herein, “in operable combination”, “in operable order” and “operably linked” refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “regulatory element” refers to a genetic element which controls some aspect of the expression of nucleic acid sequences. For example, a promoter is a regulatory element which facilitates the initiation of transcription of an operably linked coding region. Other regulatory elements are enhancers, splicing signals, polyadenylation signals, termination signals, etc.

The terms “promoter element,” “promoter,” or “promoter sequence” as used herein, refer to a DNA sequence that is located at the 5′ end (in other words precedes) the protein coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA. Promoters may be constitutive or regulatable. The term “constitutive” when made in reference to a promoter means that the promoter is capable of directing transcription of an operably linked nucleic acid sequence in the absence of a stimulus (for example, heat shock, chemicals, light, etc.). Typically, constitutive promoters are capable of directing expression of a transgene in substantially any cell and any tissue. Exemplary constitutive plant promoters include, but are not limited to SD Cauliflower Mosaic Virus (CaMV SD; see for example, U.S. Pat. No. 5,352,605, incorporated herein by reference), mannopine synthase, octopine synthase (ocs), superpromoter (see for example, WO 95/14098), and ubi3 (see for example, Garbarino and Belknap (1994) Plant Mol. Biol. 24:119-127) promoters. Such promoters have been used successfully to direct the expression of heterologous nucleic acid sequences in transformed plant tissue. In contrast, a “regulatable” promoter is one which is capable of directing a level of transcription of an operably linked nuclei acid sequence in the presence of a stimulus (for example, heat shock, chemicals, light, etc.) which is different from the level of transcription of the operably linked nucleic acid sequence in the absence of the stimulus. Promoters may be tissue specific or cell specific.

The term “cell type specific” as applied to a promoter refers to a promoter which is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, for example, immunohistochemical staining. Briefly, tissue sections are embedded in paraffin, and paraffin sections are reacted with a primary antibody which is specific for the polypeptide product encoded by the nucleotide sequence of interest whose expression is controlled by the promoter. A labeled (for example, peroxidase conjugated) secondary antibody which is specific for the primary antibody is allowed to bind to the sectioned tissue and specific binding detected (for example, with avidin/biotin) by microscopy. The term “tissue specific” as it applies to a promoter refers to a promoter that is capable of directing selective expression of a nucleotide sequence of interest to a specific type of tissue (for example, seeds) in the relative absence of expression of the same nucleotide sequence of interest in a different type of tissue (for example, leaves). Tissue specificity of a promoter may be evaluated by, for example, operably linking a reporter gene to the promoter sequence to generate a reporter construct, introducing the reporter construct into the genome of a plant such that the reporter construct is integrated into every tissue of the resulting transgenic plant, and detecting the expression of the reporter gene (for example, detecting mRNA, protein, or the activity of a protein encoded by the reporter gene) in different tissues of the transgenic plant. The detection of a greater level of expression of the reporter gene in one or more tissues relative to the level of expression of the reporter gene in other tissues shows that the promoter is specific for the tissues in which greater levels of expression are detected.

An enhancer and/or promoter may be “endogenous” or “exogenous” or “heterologous.” An “endogenous” enhancer or promoter is one that is naturally linked with a given gene in the genome. An “exogenous” or “heterologous” enhancer or promoter is one that is placed in juxtaposition to a gene by means of genetic manipulation (in other words, molecular biological techniques) such that transcription of the gene is directed by the linked enhancer or promoter. For example, an endogenous promoter in operable combination with a first gene can be isolated, removed, and placed in operable combination with a second gene, thereby making it a “heterologous promoter” in operable combination with the second gene. A variety of such combinations are contemplated (for example, the first and second genes can be from the same species, or from different species).

The term “derived from” as used herein, refers to the source of a sample, a compound or a sequence. In one respect, a sample, a compound or a sequence may be derived from an organism or particular species. In another respect, a sample, a compound or sequence may be derived from a larger complex or sequence.

The term “biodegradable” as used herein, refers to any material that can be acted upon biochemically by living cells or organisms, or processes thereof, including water, and broken down into lower molecular weight products such that the molecular structure has been altered.

The terms “transformed” refers to the introduction of foreign DNA into a cell such that the cell facilitates expression of genes encoded by the foreign DNA.

As used herein, the term “gene” means the deoxyribonucleotide sequences comprising the coding region of a structural gene and including sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogeneous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

The term “bind” as used herein, includes any physical attachment or close association, which may be permanent or temporary. Generally, an interaction of hydrogen bonding, hydrophobic forces, van der Waals forces, covalent and ionic bonding etc., facilitates physical attachment between the molecule of interest and the analyte being measuring. The “binding” interaction may be brief as in the situation where binding causes a chemical reaction to occur. That is typical when the binding component is an enzyme and the analyte is a substrate for the enzyme. Reactions resulting from contact between the binding agent and the analyte are also within the definition of binding for the purposes of the present invention.

### BRIEF DESCRIPTION OF DRAWINGS

The patent or application file contains at least one drawing executed in color. Copies of this patent or patent application publication with color drawing(s) will be provided by the Office upon request and payment of the necessary fee.

Exemplary embodiments are illustrated in referenced figures. It is intended that the embodiments and figures disclosed herein are to be consider illustrative rather than restrictive.

FIG. 1 shows exemplary polystyrene microplastic nanoparticles in solution promoting increased sedimentation of cyanobacteria strain L524 within 4 hours, increased over their parental WT strain. Amount of NPS added shown in the x-axis: 0-0.5%. Even without added NPs, L524 cyanobacteria producing limonene induced auto-sedimentation of bacteria in comparison to WT.

FIG. 2 shows exemplary majority of the polystyrene (PS) microplastics (ranging from 0-0.05%) in water were removed when L524 were added to cultivated water over wildtype (WT) bacteria. In WT cultures, large amounts of PS NPs remained in the culture water.

FIG. 3 shows exemplary microplastics (PS NPs) synergizes with limonene production for more rapid (faster) auto-sedimentation of L524 Cyanobacteria cells over time. Left, L524 (524) without PS. Right, L524 (524) with 0.05% PS (NPs).

FIG. 4A-C presents exemplary data showing representative bacteria/polymer complexes.

FIG. 4A shows exemplary Transmission Electron Microscopy (TEM) imaging of cyanobacteria WT and L524, with and without added polystyrene (PS) microplastic nanoparticles.

FIG. 4B shows exemplary Scanning Electron Microscopy (SEM) imaging of cyanobacteria WT and L524, with and without added 0.05% polystyrene (PS) microplastic nanoparticles. Engineered strains have microplastics evenly distributed among the cell surface, lower right panel, in contrast to WT showing clumps, aggregates, unevenly distributed polystyrene (PS) microplastic nanoparticles.

FIG. 4C shows exemplary plastic film produced from engineered Synechococcus elongatus UTEX 2973 (L524) cells and polystyrene NPs captured by L524. PS: 0.1 g/L polystyrene. WT & L524: 0.1 g/L (1 ml 10% polystyrene suspension) polystyrene beads (800 nm) added to 35 ml cyanobacteria (OD=15). After settling for 1 h, collect the bottom 5 ml for placement in a centrifugation (7000 rpm for 10 min). The pellets are lyophilized before adding 3 ml chloroform to each tube. Polystyrene beads and cells were dissolved with sonication (10 s on/50 s off for 6 cycles) and the solvent were centrifuged with 10000 rpm for 10 mins. The supernatants were filtrated with 0.2 um PTFE filter (need several filters for complete filtration) before casting film.

FIG. 5 shows an exemplary cyanobacteria based plastic film produced by engineered Synechococcus elongatus UTEX 2973 cyanobacteria overexpressing limonene synthase. bioplastic film composite with CMC.

FIG. 6A-D shows an exemplary method of data processing and machine learning in relation to pixel-by-pixel perdition of Light distribution patterns (LDPs) in a bioreactor.

FIG. 6A: Data pre-processing, machine learning training and prediction process. Light distribution patterns (LDPs) inside a Photobioreactor (PBR) with varied cell concentrations under different light intensities are captured and transformed to grayscale images, followed by compression to 40×18 pixels. The light intensities and cell concentrations, as well as the corresponding 40×18-pixel LDPs, are used as features and labels, respectively, in the machine learning training. In order to achieve accurate prediction, the training and prediction are performed pixel by pixel.

FIG. 6B shows an exemplary Pixel-by-pixel R² evaluation of LDPM prediction over testing samples suggesting LDPM performs well at majority of pixels. Evaluation over all pixels on testing LDPs showed an R² score of 0.993.

FIG. 6C further verifying the accuracy of the LDPM.

FIG. 6D shows an exemplary schematic graph of linear regression showing near-linear correlation between GSV and light intensity across all cell concentration (average R² score at 0.969), suggesting the grayscale value is a legitimate representation of light intensity. Cell concentrations from left to right are 0.11973, 0.21294, 0.40872, 0.45162, 0.54405, 0.62712, 0.74256, 0.82056, 0.90948, 0.96915, 1.10604, 1.2246, 1.3026, 1.3923, 1.443, 1.5444, 1.7901, 1.9188, 2.0241, 2.3556, 2.535, 2.9601, 3.6777 g/L.

FIG. 7A-G shows an exemplary schematic of Growth rate prediction, growth simulation and semi-continuous algal cultivation (SAC).

FIG. 7A: Overview of schematic workflows as methods for growth simulation. The growth simulation could be achieved by integrating the LDPM with an additional growth rate prediction model, GRM.

FIG. 7B: Overview of schematic workflows as methods for GRM training. The LDP features predicted by LDPM and corresponding growth rates calculated from growth curves were used as features and labels.

FIG. 7C: The accuracy of GRM prediction was evaluated with an R score of 0.992, indicating high precision.

FIG. 7D: The biomass productivities sustain at around 2 g/L/day over 7 days in SAC (d, SC) while they decreased over time in fed-batch (FB).

FIG. 7E: The cyanobacterial growth (biomass production) with different initial OD under low light were simulated (lines) and monitored (columns). Original data points are shown with stars. Data are presented as mean values +/−standard deviations (n=3 independent samples with 3 technical replicates).

FIG. 7F: The cyanobacterial growth (biomass production) with different initial OD under high light were simulated (lines) and monitored (columns). Original data points are shown with stars. Data are presented as mean values +/−standard deviations (n=3 independent samples with 3 technical replicates)

FIG. 7G: The cyanobacterial growth (biomass production) with different initial OD under changing light were simulated (lines) and monitored (columns). Original data points are shown with stars. Data are presented as mean values +/−standard deviations (n=3 independent samples with 3 technical replicates)

The similar trends between simulations and measurements verified the accuracy of the simulation and legitimized the simulation as a reliable tool to inform algal cultivation system development. After light condition optimization, biomass productivity from machine learning-informed SAC was evaluated, with fed-batch as control.

FIG. 8A-J shows an exemplary limonene production enables cell aggregation in UTEX 2973.

FIG. 8A: Cell aggregation is not observed in wild-type (WT) cells.

FIG. 8B: Cell aggregation is observed in L524 cells.

FIG. 8C: Quantification analysis suggests 91% of L524 cells are found in aggregates. Original data points are shown on the plot with dots. Data are presented as mean values +/−standard deviations (n=3 independent samples with 3 technical replicates).

FIG. 8D: Showing a lack of limonene droplets on WT cells as shown by TEM images.

FIG. 8E: Limonene droplets are found on L524 cells as shown by TEM images.

FIG. 8F: L524 limonene production is about 1.4 mg/L/day/OD₇₃₀ in normalized productivity as determined by GC-MS. SRS chemical imaging was used to identify chemical compositions in the droplets. Original data points are shown on the plot with dots. Data are presented as mean values +/−standard deviations (n=3 independent samples with 3 technical replicates).

FIG. 8G: Limonene signal from WT cells.

FIG. 8H: Limonene signal from L534 cells which is significantly higher that found in L524. This differential is more evident at the cell surface, where limonene droplets attach to the outer cell surface.

FIG. 8I: Surface-attaching limonene form inter-cell junctions bridging cells thereby promoting cell aggregation.

FIG. 8J: Cell surface hydrophobicity (BATH assay) was significantly reduced in L524 cells that suggests over 40% of L524 cells bind to hydrophobic hydrocarbon, confirming their increased cell surface hydrophobicity. Scale bar, 2 pm. N.D., not detected. **, p=8.4×10⁻¹⁰ (two-tailed Student's t test, n=3 independent samples with 3 technical replicates). Original data points are shown on the plot with dots. Data are presented as mean values +/−standard deviations (n=3 independent samples with 3 technical replicates).

FIG. 9A-C shows an exemplary Evaluation of Aggregation-Based Sedimentation (ABS).

FIG. 9A: L524 cells after 1-hour sedimentation with wild-type as control.

FIG. 9B: L524 cells started to settle within 5 minutes and over 75% cells were settled after 15 minutes, suggesting the high settling velocity of ABS.

FIG. 9C: Vertical cell concentration analysis suggests that 85% and 93% cells settled to the bottom of the harvesting vessel (20 cm in depth) within 30 minutes and 6 hours, respectively (b). Moreover, the biomass concentration at the bottom reaches 357 OD (139.2 g/L), which delivers 14% solids content as an output for ABS. Original data points are shown on the plot with red (0.5 h) or blue (6 h) stars. Data are presented as mean values +/−standard deviations (n=3 independent samples with 2 technical replicates).

FIG. 10A-D shows an exemplary sustainable and higher limonene and biomass productivities achieved in SAC (SC). Data are presented as mean values +/−standard deviations (n=3 independent samples).

FIG. 10A: Concept figure shows the integration of machine learning-informed SAC and ABS for biofuel production.

FIG. 10B: Sustainable limonene production at around 5 mg/L/day as compared to batch cultivation (BA) which reaches a Day 2 plateau due to nutrient depletion and light limitation (FB).

FIG. 10C: Sustainable biomass production at about 2.2 g/L/day as compared to batch cultivation (BA) which reaches a Day 2 plateau due to nutrient depletion and light limitation (FB).

FIG. 10D: Growth of cyanobacteria with batch (BA), fed-batch (FB) and SAC (SC).

FIG. 11A-F shows an exemplary scaling-up of SAC with a pond system.

FIG. 11A: A pond system design for evaluation of an adapted LDPM.

FIG. 11B: The LDPM achieved an overall R² score of 0.986 and,

FIG. 11C: Pixel-by-pixel analysis suggested that prediction at all pixels were reasonably good with a minimal R² score of 0.943.

FIG. 11D: Biomass production with the outdoor pond system achieved a record biomass productivity.

FIG. 11E: Prediction evaluation of the GRM adapted for a pond system.

FIG. 11F: Machine learning-based growth simulation (line) suggested that setting the initial cell concentration to around 0.4 g/L achieves optimal biomass productivity under the growth condition mimicking Texas Summer, which was well supported by the measured results (bars). Original data points are shown on the plot with dots. Error bands represent standard deviations (n=2 independent samples).

FIG. 12 shows an exemplary map and sequence of pWX1118.

FIG. 13 shows an exemplary Pixel-by-pixel evaluation of LDPM prediction over testing samples. 94.4% of pixels achieved R² values higher than 0.90, and 0.8% of pixels had R² values in the range of 0.79 to 0.85.

FIG. 14A-D shows an exemplary Light intensity (represented by GSV) changes over the length of the light path. GSVs in the middle column (column 20, row 1-18) of LDPs were extracted to represent light intensities over light paths and plotted against distances from light sources. Different colors in the figures represent different intensities of light sources (107, 178, 267, 357, 570, 714 μmol m⁻² s⁻¹).

FIG. 14A: Light intensity decreased slightly over the path when cell concentration was low.

FIG. 14B: Significant decreases in light intensity were observed when cell concentration increased.

FIG. 14C: Significant decreases in light intensity were observed when cell concentration increased.

FIG. 14D: Light intensity dropped sharply (GSV below 20 within 1 cm) when cell concentration reached 2.9601 g/L. The results suggest intensified mutual shading at higher cell concentration.

FIG. 15A-F shows an exemplary relationship between cell concentration (OD₇₃₀), dark area derived from LDP, and growth rate. Slopes of growth curves were calculated to represent growth rates at different light intensities. LDPs over the growth at given light conditions were predicted by LDPM and dark areas were defined as LDP pixels with GSVs less than 25.5. Growth rates peaked at 36.8±4.7 hours and dark areas reached 43.1±4.9% regardless of light intensity. Overall, three stages were identified: i) a zeros stage (left of the first dashed line); ii) an increasing stage (in between the first and second dashed lines) which overlapped significantly with the fastest growth period; and iii) a plateau stage (right of the second dashed line).

FIG. 15A: Growth curves (blue) were generated by fitting cell concentration (OD₇₃₀ and time) collected from cultivations under light intensities at 107 μmol m⁻² s⁻¹.

FIG. 15B: Growth curves (blue) were generated by fitting cell concentration (OD₇₃₀ and time) collected from cultivations under light intensities at 178 μmol m⁻² g⁻¹.

FIG. 15C: Growth curves (blue) were generated by fitting cell concentration (OD₇₃₀ and time) collected from cultivations under light intensities at 267 μmol m⁻² g⁻¹.

FIG. 15D: Growth curves (blue) were generated by fitting cell concentration (OD₇₃₀ and time) collected from cultivations under light intensities at 357 μmol m⁻² g⁻¹.

FIG. 15E: Growth curves (blue) were generated by fitting cell concentration (OD₇₃₀ and time) collected from cultivations under light intensities at 570 μmol m⁻² s⁻¹.

FIG. 15F: Growth curves (blue) were generated by fitting cell concentration (OD₇₃₀ and time) collected from cultivations under light intensities at 714 μmol m⁻² g⁻¹.

FIG. 16A-H shows an exemplary validation of growth simulation by machine-learning models under different growth conditions and comparison between semi-continuous algal cultivation (SAC) and fed-batch. Data are presented as mean values +/−standard deviations (n=3 independent samples).

FIG. 16A: The growth simulation at a light intensity of 107 m⁻² s⁻¹ achieved R² scores of 0.996 indicating a high prediction accuracy.

FIG. 16B: The growth simulation at a light intensity of 178 m⁻² s⁻¹ achieved R² scores of 0.999 indicating a high prediction accuracy.

FIG. 16C: The growth simulation at a light intensity of 267 m⁻² s⁻¹ achieved R² scores of 0.996 indicating a high prediction accuracy.

FIG. 16D: The growth simulation at a light intensity of 357 m⁻² s⁻¹ achieved R² scores of 0.998 indicating a high prediction accuracy.

FIG. 16E: The growth simulation at a light intensity of 570 m⁻² s⁻¹ achieved R² scores of 0.996 indicating a high prediction accuracy.

FIG. 16F: The growth simulation at a light intensity of 714 m⁻² s⁻¹ achieved R² scores of 0.997 indicating a high prediction accuracy.

FIG. 16G: The growth simulation at a light intensity of 178-714-178 m⁻² s⁻¹ achieved R² scores of 0.978 indicating a high prediction accuracy.

FIG. 16H: Growth comparison of UTEX 2973 with SAC and fed-batch cultivation.

FIG. 17A-B shows an exemplary GRM adapted for growth rate prediction with double light sources.

FIG. 17A: Validation of the growth rate prediction by the GRM adapted for double light.

FIG. 17B: Growth simulation suggests setting initial OD₇₃₀ at 2.3 delivers highest biomass productivity.

FIG. 18 shows an exemplary transmission electron microscopy (TEM) reveals cell surface differences between Synechococcus elongatus PCC 7942 and UTEX 2973. UTEX 2973 showed a relatively smooth cell surface and more formed pili as compared to PCC 7942. Similar results were found in two independents observations.

FIG. 19 shows an exemplary comparison of growth between UTEX 2973 WT and L524. No significant growth differences were found between WT and L524 in the given growth conditions. Data are presented as mean values +/−standard deviations (n=3 independent samples).

FIG. 20 shows an exemplary standard curve used to normalize limonene productivity with recovery rate. Limonene concentrations of 250, 500, 750, 1000 μg/mL were used to spike the UTEX 2973 wildtype cells. Limonene was collected and measure as described in the Methods of the main text. Data are presented as mean values +/−standard deviations (n=3 independent samples).

FIG. 21 shows an exemplary techno-economic analysis of the pond SAC platform. The NREL algae farm model projects a MBSP of approximately $281 per ton based on the outdoor trial yield. Cost breakdown suggests the dewatering process accounts for $24.50 per ton.

FIG. 22A-B shows an exemplary LDP prediction for open pond system and conversion between turbidity and OD₇₃₀.

FIG. 22A: A process of adapting LDPM for pond system prediction.

FIG. 22B: A calibration curve for OD-turbidity (Attenuation Unit, AU) conversion.

FIG. 23A-E present illustrations of a representative synthetic biology design to drive limonene biosynthesis, alter cell hydrophobicity and enable efficient sedimentation.

FIG. 24A-F present exemplary data showing the verification of MP removal capacity by the limonene producing strain using an even suspension of polystyrene nanoparticles.

FIG. 25A-C present exemplary data showing intracellular and extracellular interactions that mediate microplastics bioremediation by L524 cells overexpressing limonene.

FIG. 26A-N present exemplary data showing that cyanobacteria overexpressing limonene form molecular interactions with polyethylene (PE) and polyethylene terephthalate (PET) sufficient for bioremediation.

FIG. 27A-G present exemplary data showing a representative MP upcycling scheme for remediated bioplastic composite production.

### DESCRIPTION OF INVENTION

The present invention is related to the fields of bioplastics. In particular, a strain of photosynthetic cyanobacteria (i.e. blue-green algae) having a high growth rate was engineered to overexpress limonene synthase. These engineered cyanobacteria improve auto-sedimentation for biomass harvest and provide for plastic bioremediation capture. Further, machine-learning methods are provided for improving some of the aspects of cultivating cyanobacteria including, but not limited to, assessing real-time light availability and predicting real-time algae growth rates to increase a cyanobacteria biomass.

In some embodiments, the present invention contemplates methods to improve algal biomanufacturing, algal bioproduction and/or renewable algal-based fuels and products.

In one embodiment, the present invention contemplates a method utilizing artificial intelligence (AI) techniques to guide algal cultivation design (e.g., machine learning). In particular, the data provided herein suggest quantitative insights into how light intensities and cell density shape LDPs and how LDPs, in turn, impact cyanobacterial growth rates. For example, an integration of LDPM and GRM enables reliable simulation of growth curves based on initial OD and light intensity. This knowledge permits the development of semi-continuous algal cultivation (SAC) systems at a precisely defined the optimal initial OD that achieves maximized growth. The high accuracy, broad prediction range, and superior capacity to handle complexity of machine learning models produced broader adaptability in constant or changing light and in indoor/outdoor PBRs or pond systems. The principle and design of the study can be broadly applied. The machine learning models themselves can be broadly adapted to different to industrial microbiology and biomanufacturing techniques to guide algal cultivation management and design. The models proposed herein can be further optimized to integrate nutrient, temperature and other factors to achieve even broader adaptability.

In one embodiment, the present invention contemplates a method which achieves aggregation-enabled sedimentation (ABS) by manipulating cyanobacterial cell hydrophobicity. Self-sedimentation achieved a high solids load and enabled a efficient and low-cost harvest method for algal bioproduction, overcoming a major challenge in the algal industry. Furthermore, the principle can be used to design ABS in other species for broader biomanufacturing applications.

In one embodiment, the present invention contemplates a method which achieves increased yields of biomass in both indoor/outdoor PBR and pond systems as compared to conventional techniques. For example, an outdoor raceway pond productivity achieved 43.3 g/m²/d, which surpasses the U.S. DOE 2022 target by 1.7 times. The consistency of outdoor productivity and indoor estimated productivity (43.3 g/m²/d vs. 48.1 g/m²/d) again proves the effectiveness and reliability of the inventive embodiments as described herein. Due to enhanced yields and reduced operating costs by ABS, SAC may achieve an economical algal bioproduction below $300 per ton. Furthermore, the lower cost of algal biomass enables economically competitive applications in broader industries, including algal biofuel, animal feed, food additives, and various specialty products^{47, 48, 49, 50}.

In one embodiment, an isolated automated device and/or system are contemplated. In one embodiment, a system comprises collection lines, with tubes inserted into the water, including pond water for programmed removal of water samples for counting cyanobacteria for biomass estimates, e.g., pond water, light measuring panels, etc. for monitoring growth conditions of the algae, and perhaps other methods of predictions, with a wireless connection for sending data to sight smart phones or computers for analysis, Analysis comprises any of the methods of prediction and growth measurement, as described herein.

In particular, a strain of photosynthetic cyanobacteria having a high growth rate was engineered for overexpressing limonene synthase. The engineered bacteria enable auto-sedimentation for biomass harvest. Further, methods are provided for improving some of the aspects of cultivating, including but not limited to methods for automated semi-continuous algal cultivation (SAC); accessing real-time light availability inside an algal culture with machine learning based light distribution prediction models (LDPM); predicting real-time algae growth rate with Growth Rate Prediction Model (GRM); informing cultivation design with growth simulation based on the LDPM and GRM; scaling up the machine learning-informed SAC production within bodies of water such as ponds, lakes and the like; for increasing cyanobacteria biomass. In one embodiment for improving aspects of cultivating and harvesting algae, such methods comprise photosynthetic cyanobacteria having a high growth rate overexpressing limonene synthase. On the other hand, methods are provided for plastic nanoparticle capturing by aforementioned limonene producing cyanobacterial strain. The captured nano plastics, together with cyanobacterial cells can be used for plastic film production. Further, biomass produced from the machine learning-informed SAC can be used as a composite enforcer for biobased plastic films.

It is not intended to limit parameters to environments similar to Texas, although Texas, United States, was used as an exemplary system. In fact, use of such methods described herein would change parameters for each different pond growth-harvest cycles at different times of the year, including Texas, other States in the US, territories, etc., and in international locations.

The L524 cells can aggregate and settle by themselves and the presence of NPs is not required for sedimentation. We took advantage of the self-aggregation of L524 for simplified harvesting and NPs were not used for this purpose. Instead, we demonstrated the capacity of capturing NPs as an additional application, e.g. embodiment.

In particular, a strain of photosynthetic cyanobacteria having a high growth rate was engineered for overexpressing limonene synthase. The engineered bacteria enable auto-sedimentation for biomass harvest. For example, engineered Synechococcus elongatus UTEX 2973 cells enable auto-sedimentation for cyanobacterial biomass harvest. Further, methods are provided for improving some of the aspects of cultivating, including but not limited to methods for automated semi-continuous algal cultivation (SAC); accessing real-time light availability inside an algal culture with machine learning based light distribution prediction models (LDPM); predicting real-time algae growth rate with Growth Rate Prediction Model (GRM); informing cultivation design with growth simulation based on the LDPM and GRM; scaling up the machine learning-informed SAC within ponds; etc. for increasing cyanobacteria biomass. In one embodiment for improving aspects of cultivating and harvesting algae, such methods comprise photosynthetic cyanobacteria having a high growth rate overexpressing limonene synthase. On the other hand, methods are provided for plastic nanoparticle capturing by aforementioned limonene producing cyanobacterial strain. The captured nano plastics, together with cyanobacterial cells can be used for plastic film production. Further, biomass produced from the machine learning-informed SAC can be used as a composite enforcer for biobased plastic films. Methods including production of biomass, etc., are within bodies of water such as ponds, lakes, bioreactors and the like, as described herein.

Algal biofuel is regarded as one of the ultimate solutions for renewable energy, but its commercialization is hindered in part by high harvest costs and growth limitations caused by mutual shading. Terpenoids are a large class of natural products with diverse biological functions, including photon harvesting (e.g., chlorophylls), membrane stability (e.g., sterols), and multi-trophic signaling. Many terpenoids are also valuable chemicals with broad applications in the pharmaceutical, nutraceutical, cosmetic, and biofuel industries. Limonene as one example of a terpenoid, is regarded as a potential component of jet fuel, and as a fuel additive that enhances cold-weather performance, among other potential uses of limonene as produced herein. Isolated and purified limonene, as described herein, may be used as part of food, pharmaceuticals, cosmetics, used as hand cleaners, industrial cleaners, degreasers, etc.

In part to solving high harvest costs, as described herein, a cyanobacteria UTEX 2973 WT strain having a high growth rate (a growth rate much higher than Synechococcus sp. PCC 7942) was engineered to produce a higher amount of limonene than UTEX 2973 WT, resulting in both intracellular and extracellular limonene in the engineered UTEX 2973 L524 strain, i.e. L524. Extracellular limonene may be harvested, and purified to use for a variety of applications. Moreover, extracellularly expressed limonene attached to the cell surface of L524 bacteria in turn induced attachment of the L524 bacteria to each other, without being limited to any particular mechanism. Without being limited to any particular mechanism, it is believed that aggregation of Synechococcus elongatus UTEX 2973 cells and engineered Synechococcus elongatus UTEX 2973 cells (e.g., L524) drive efficient cell sedimentation and may be used for cost-effective biomass harvesting.

The presently disclosed invention has multiple advantages over conventional systems under the following exemplary technological perspectives:
1. A model was developed which identified optimal optical density (OD) for initial inoculation under minimal light penetration conditions, e.g. 2.3 OD₇₃₀. Semi-continuous and continuous cultivation based on a defined OD to remove liquid containing cyanobacteria and maximize light penetration. An advantage of using cultivation methods described herein is for maximizing light penetration into cultures is by removing cyanobacteria and restarting culture at an approximate 2.3 OD at wavelength 730 (OD₇₃₀). Thus allowing maximal use of a body of water for culture.
2. A novel engineered strain of cyanobacteria was produced. In particular, an engineered Synechococcus elongatus UTEX 2973 over-expressing limonene for inducing auto-sedimentation. Engineering a strain of cyanobacteria (i.e., Synechococcus elongatus UTEX 2973) for expressing limonene to lead to strong auto-sedimentation (regular cyanobacteria not expressing limonene does not auto-sediment) and without adding microplastics to the solution. While engineering limonene expression in cyanobacteria was described in Wang, 2016, it was not shown that expression of limonene led to auto-sedimentation. This is because: i) Limonene creates hydrophobic surface; and ii) the cell surface of engineered Synechococcus elongatus UTEX 2973 is flat and can allow hydrophobic surface to aggregate and lead to sedimentation. The development of cyanobacteria for expressing limonene leads to low-cost harvesting and use for commercialization. An advantage of using engineered Synechococcus elongatus UTEX 2973 is for inducing auto-sedimentation. Engineering a strain of cyanobacteria (i.e., Synechococcus elongatus UTEX 2973) for expressing limonene to led to removal of microplastics from a liquid solution. The development of cyanobacteria for expressing limonene leads to low-cost harvesting and use for commercialization. An advantage of using engineered Synechococcus elongatus UTEX 2973 is for removing microplastics from solution.
3. Using auto-sedimentation, a semi-continuous cultivation method maximized yield.
4. Auto-sedimentation was shown to remove microplastics from water.
5. Microplastics were observed to synergize auto-sedimentation.
6. Cyanobacteria were mixed with modified carbohydrates to produce a plastic film. Cyanobacteria to produce bioplastics, when mixing with CMC. An advantage of using engineered Synechococcus elongatus UTEX 2973 is producing a bioplastic, e.g., a carbohydrate-based film, e.g., CMC. e.g., CMC.

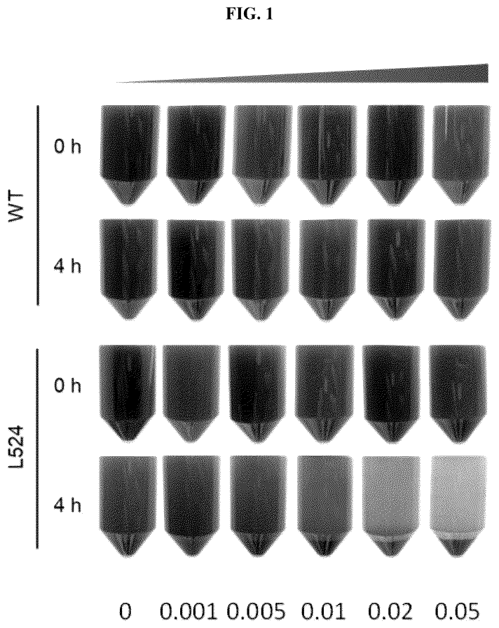

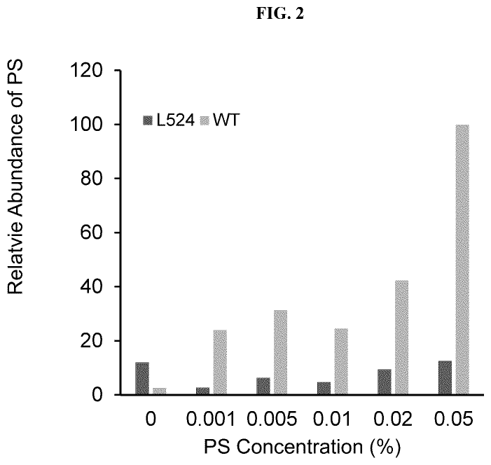

Although Synechococcus elongatus UTEX 2973 cells auto-sediment, engineered strains of Synechococcus elongatus UTEX 2973 cells producing limonene auto-sediment faster than parental Synechococcus elongatus UTEX 2973 cells. An engineered, Synechococcus elongatus UTEX 2973 cells, i.e., L524 strain, producing limonene for causing faster auto-sedimentation can also be used for removing the microplastics from water. As described herein, and shown in exemplary figures, the addition of microplastic NPs, improves the sedimentation speed of L524. FIG. 1 shows polystyrene microplastics promotes the sedimentation of L524 strain. Furthermore, FIG. 2 shows the removal of the microplastics by L524 strain via auto-sedimentation. FIG. 2 The majority of the polystyrene microplastics was removed in L524 cultivated water. Furthermore, the L524 strain actually synergize with microplastics to accelerate the sedimentation of the cells. FIG. 3 Microplastics synergizes for more rapid L524 cell sedimentation. Examples of nanoparticles added to solutions include polystyrene NPs, and the like. Percentages of NPs added to solutions range from 0%, 0.01%, up to 0.05%, up to 1% or more. NPs added to solution in the presence of rapidly growing cyanobacteria overexpressing limonene synthases result in faster production of concentrated biomass, bioplastic-based films, etc. from auto-sedimentation.

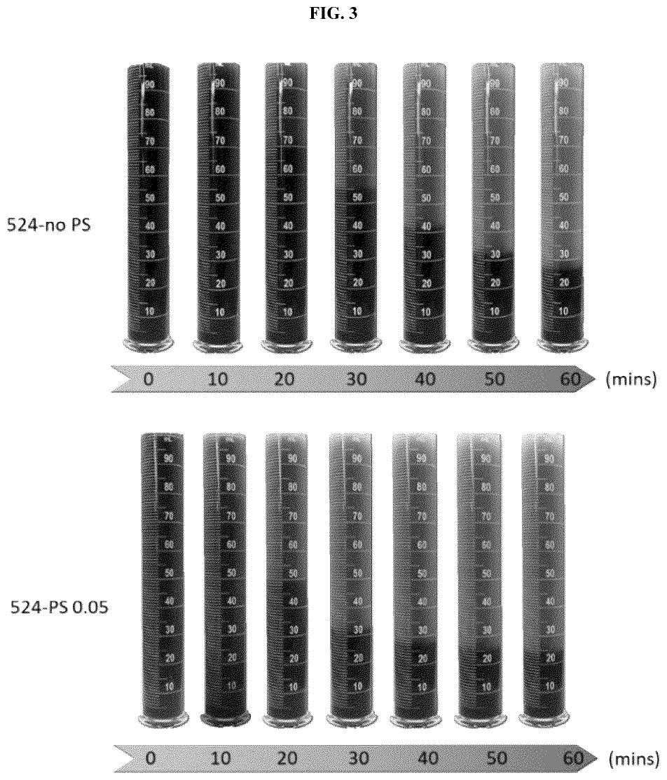

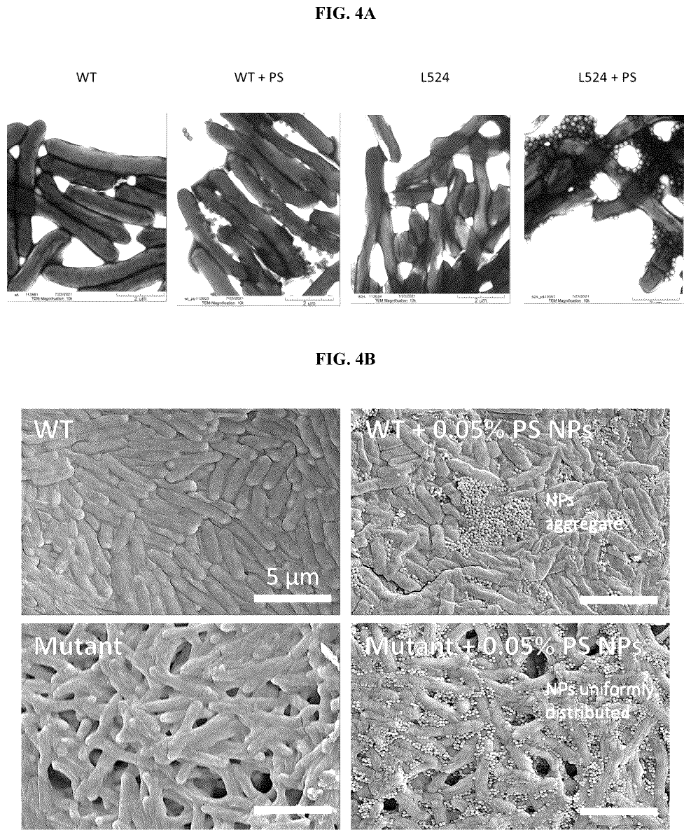

Imaging analysis further verified that hydrophobic microplastic were attached to the cell surface through hydrophobic interaction with the limonene enriched cell surface. FIG. 4A TEM imaging of the cyanobacteria together with microplastics. Furthermore, the FIG. 4B SEM image also confirmed that the microplastics were distributed evenly among the cell surface in the engineered strain, due to hydrophobic interactions. However, such interaction is not observed in the WT strains. FIG. 4B Engineered strains have microplastics evenly distributed among the cell surface. Moreover, cyanobacteria cell was used to make plastic film when using with chloroform. FIG. 4C shows an exemplary cyanobacteria-based plastic (bioplastic) film. See brief description of plastic film formation herein.

In addition, UTEX 2973 and engineered strain was directly used as a composite enforcer to make biobased plastic films when using with carboxymethyl cellulose (CMC). FIG. 5 shows an exemplary cyanobacteria-based plastic (bioplastic) film.

### I. Engineered Cyanobacterial Strains

Altering cell surface hydrophobicity achieved more efficient cell aggregation. Despite the potential of SAC, its feasibility depends heavily on cost-effective harvesting, a major challenge in algal biofuel. Sedimentation or auto-flocculation represent an ideal method for cyanobacterial biomass harvesting^{3, 4, 5}, but auto-flocculation and sedimentation without chemical or microorganism additions remain challenging for single-cell algae. According to Stokes' Law, sedimentation rate is determined by the size and density of particles.³ UTEX 2973 cells contain around 42.8% protein, 36.5% carbohydrates, and merely 11.2% lipid. Due to the high carbohydrate content (average density around 1500 kg/m³), high protein content (average density around 1300 kg/m³), and low lipid content (average density around 860 kg/m³) of UTEX 2973 cells,³ they should be dense enough for sedimentation in water (˜1000 kg/m³). We suspected that auto-flocculation or sedimentation of UTEX 2973 might be achieved by increasing particle size via cell aggregation. Synechococcus elongatus UTEX 2973 is a close relative of the widely studied cyanobacterium Synechococcus elongatus PCC 7942, an organism that grows more than two times slower. A small number of nucleotide changes are differences between the genomes of these two cyanobacterial strains. Yu, et al., Synechococcus elongatus UTEX 2973, a fast growing cyanobacterial chassis for biosynthesis using light and CO₂. Sci Rep-Uk 5, (2015).

One approach to achieve cell aggregation is to increase cell surface hydrophobicity to promote cell-to-cell self-adhesion.²⁸ We hypothesized that engineering hydrophobic molecule production could increase cell hydrophobicity and drive cell aggregation for sedimentation.

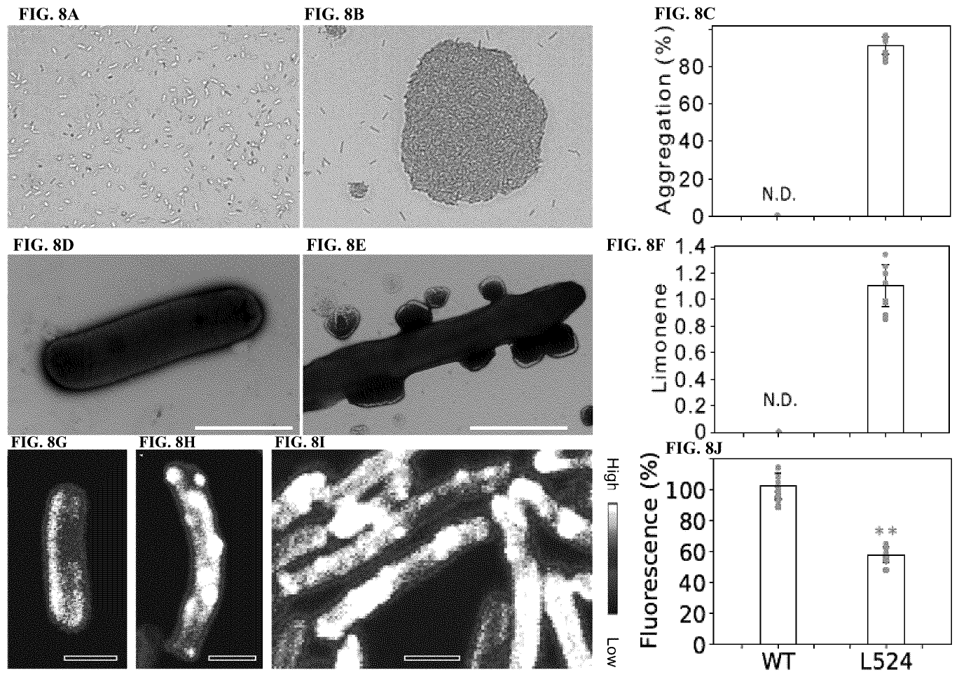

To test this hypothesis, we overexpressed a limonene synthase in UTEX 2973 to produce limonene, a strong hydrophobic terpene that can be excreted from cyanobacterial cells.^{29, 30, 31} The strain was named L524. A cell aggregation study showed that aggregation occurred in L524 (FIG. 8B), but not in the wild-type (FIG. 8A). Quantitative analysis demonstrated that 91% of L524 cells aggregated after 30 minutes (FIG. 8C).

To further understand if the aggregation resulted from limonene production, we observed L524 cells under Transmission Electron Microscopy (TEM) and verified the limonene production by gas chromatography-mass spectrometry (GC-MS). Putative limonene droplets were found on L524 cells (FIG. 8E) but not on wild-type cells (FIG. 8D). The formation of the droplets might be a process for limonene to secrete from cells. Indeed, limonene production was detected by GC-MS in L524 at around 1.4 mg/L/day/OD₇₃₀ (FIG. 8F).

To further verify the accumulation of limonene in L524 cells, stimulated Raman scattering (SRS) microscopy was used to visualize limonene distribution in cyanobacterial cells.^{32, 33} As shown in FIG. 8G-I, the weak signal from wild-type cells (FIG. 8G) can be considered background since limonene production was not detected by GC-MS in the wild-type (FIG. 8F). By contrast, strong limonene signals were observed in L524 cells, primarily presenting as droplets (FIG. 8H). These results support the hypothesis that droplets found on the L524 cell surface by TEM were composed of limonene. SRS imaging on L524 aggregates showed the presence of limonene at cell junctions (FIG. 8I), indicating a role of limonene droplets in mediating aggregation.

Limonene could promote aggregation in three ways. First, hydrophobic limonene molecules could directly increase cell surface hydrophobicity, which was supported by a bacterial adherence to hydrocarbon (BATH) assay³⁴. While almost all wild-type cells stayed in the aqueous phase in the assay, over 40% L524 cells adhered to hydrocarbon as demonstrated by reduced chlorophyll fluorescence in the aqueous phase (FIG. 8J). Such hydrophobicity increases could be the driving force for cell aggregation. Second, once cells are close enough, droplets on cell surfaces could fuse to further enhance cell-to-cell adherence (FIG. 8I). Third, while a uniform negatively charged cell surface is critical to maintain cell suspension,^{3, 35} the neutral limonene could disrupt cell surface charge and contribute to aggregation.

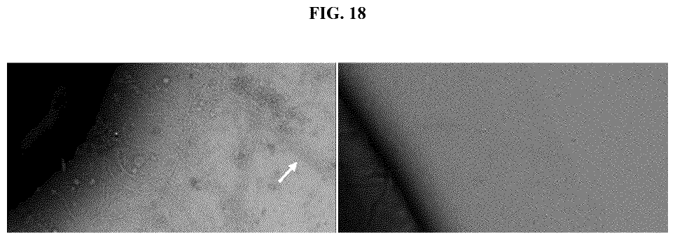

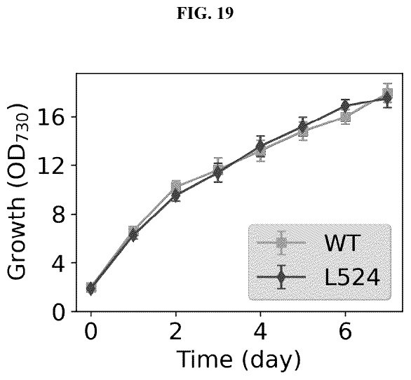

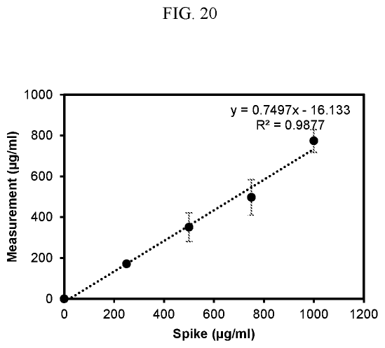

Moreover, the unique ‘smooth’ cell surface of UTEX 2973 might also promote aggregation in combination with the hydrophobic interaction of limonene production. Unlike other cyanobacteria, pili rarely form on the UTEX 2973 cell surface, presumably due to the early termination of the pilN protein³⁶. The flatter cell surface of UTEX 2973 allows limonene droplets among different cells to interact with one another more easily compared to strains like PCC 7942. See, FIG. 18. Together, limonene production and the smooth cell surface might have enabled the engineered cells to aggregate due to hydrophobic interaction in a 5 water environment.

Limonene production by L524 from SAC surpassed previously reported yields as shown in Table 1.

TABLE 1
Recent publications about limonene production in cyanobacteria.

| | Productivity | Yield | Time | |
|---|---|---|---|---|
| Strain | (mg/L/d) | (mg/L) | (d) | Reference |
| Anabaena PCC 7120 | 0.1 * | 0.2 | 2 | ⁵⁴ |
| Synechococcus PCC 7942 | 0.8 * | 2.5 * | 5 | ²⁹ |
| Synechocystis PCC 6803 | N.A. | 6.7 | 7 | ³¹ |
| Synechococcus PCC 7002 | 1.5 * | 4 | 4 | ³⁰ |
| Synechococcus UTEX 2973 | 5.0 | 50 | 11 | This study |

* The value is estimated from figures.
N.A., Not applicable.

The high daily productivity could be attributed to the optimal light availability and the high photosynthetic capacity of UTEX 2973.^{26, 27} High yields highlight the strength of SAC in maintaining algal bioproduction at optimal rates over an extended period. A detailed comparison of productivity on the seventh day showed an approximate 6-fold difference in limonene productivity between SAC and batch cultivation. Similarly, biomass production is compared between these relevant studies using PBRs. See, Table 2.

TABLE 2
Select publications on cyanobacterial biomass
production with Photobioreactors (PBRs).

| | PBR Size | Yield | Time | |
|---|---|---|---|---|
| Strain | (cm) | (g/L) | (d) | Reference |
| S. elongatus PCC 11801 | 3 ^{#} | 2 * | 3 | ⁵⁵ |
| S. elongatus PCC 11802 | 3 ^{#} | 3 * | 5 | ⁵⁶ |
| S. elongatus BDU 130192 | 3 ^{#} | 2 | 4 | ⁵⁷ |
| S. elongatus PCC 7942 | 3 ^{#} | 1.9 * | 5 | ⁴² |
| S. elongatus UTEX 2973 | 3 ^{#} | 2.3 * | 5 | ⁴² |
| S. elongatus UTEX 2973 | 10 × 5 | 23.4 | 11 | This study |

^{#}Diameter of the cylinder PBRs.
* The value is estimated from figures.

Although one study showed higher algal biomass productivity, the study was carried out in shaking flasks with very small volume and the addition of costly Vitamin 12 (thus not included in the comparation).⁴³ We have achieved comparable biomass productivity with cultivation systems that are 20-times larger in volumes than the study. Overall, this study presented significant improvements in algal bioproduction by machine learning-informed SAC, where mutual shading has been overcome and harvesting costs substantially reduced by synthetic biology-enabled ABS.

A construct, pLB524, was used to create the strain L524 via homologous recombination. To build pLB524, homologous sequences of UTEX 2973 neutral site I and limonene synthase were amplified from pWX1118, as described in Wang, et al. Enhanced limonene production in cyanobacteria reveals photosynthesis limitations. Proc Natl Acad Sci USA 113, 14225-14230 (2016), herein incorporated by reference in its entirety. Primer pairs of NS-DS-F/NS-US-R were used. See, Table 3.

TABLE 3
Exemplary Primers

| Primer | | |
|---|---|---|
| name | Sequence taggacgga | Note |
| NS-DS-F | cacgaggccctttcgtcttcaagaaATGGATCTGACCAACATG | Building L524 |
| | (SEQ ID NO: 2) | |
| NS-US-R | atcgatgataagctgtcaaacatgagaaAAACGCGCGAGGCAGGAT | Building L524 |
| | (SEQ ID NO: 3) | |
| NSI-F | TCAGCTGCTTTAGGCCCACCAGTTTGAAG (SEQ ID | Segregation |
| | NO: 4) | |
| NSI-R | TTATCTCTCGGCTAGTGGACGCAAGCAGCG (SEQ ID | Segregation |
| | NO: 5) | |
| petB1-F | CGACTGGTTCGAGGAGCGTC (SEQ ID NO: 6) | qRT-PCR, IS |
| petB1-R | TTGCAAAGCCGGTGGCAAAC (SEQ ID NO: 7) | qRT-PCR, IS |
| LS1-F | CTCGAATCTGCCCGCGAGTT (SEQ ID NO: 8) | qRT-PCR, LS |
| LS1-R | GATCCAGACCGGGGCATTGG (SEQ ID NO: 9) | qRT-PCR, LS |

IS, internal standard; LS, limonene synthase.

The amplified fragment was then integrated into pBR322 by Gibson assembly. The assembled pLB524 was transformed into UTEX 2973 WT by conjugation^{25, 36}. Briefly, a cargo E. coli strain containing pLB524 and helper plasmid pRL623 was first mixed with conjugal strain containing pRL443 for 30 min at 37° C., before mixing with WT UTEX 2973 cells. The mixture was then incubated on BG11+5% LB plates without antibiotics and then transferred to BG11 plates with 5 μg/ml spectinomycin/streptomycin. Transformants that had been segregated with increasing antibiotics (5 pg/ml, 10 μg/ml, and 15 μg/ml) for three rounds were verified by PCR and further confirmed by qPCR with primers provided in Table 3. It is not intended to limit LS overexpression, to spearmint LS, indeed, other LS genes may be codon optimized for S. elongatus and expressed WT UTEX 2973 cells.

Spearmint (Mentha spicata) Limonene synthase (LS) was codon optimized for S. elongatus expression from a codon optimized derived from plasmid pWX1118, truncated to remove the plastid signal peptide. SEQ ID NO: 1 below was overexpressed in UTEX 2973 to produce L524 cells:

GATCTCAATGAATATTGGTTGACACGGGCGTATAAGACATGTTATACTG
TTGAATAACAAGTTTACCGTTCCCAAAAATAAAGAAGGAGGAACAGCAT
GCGACGCTCCGGCAATTATAATCCGTCTCGCTGGGATGTTAATTTCATC
CAGTCCCTCTTGTCGGACTACAAAGAGGACAAACATGTCATCCGGGCCA
GCGAGCTGGTCACACTGGTTAAAATGGAATTGGAGAAAGAGACGGACCA
GATTCGACAGCTGGAGCTCATTGACGATTTGCAACGCATGGGACTCTCG
GATCATTTCCAGAACGAATTTAAAGAGATCCTGTCTAGCATTTACCTGG
ACCACCATTACTATAAGAACCCATTCCCTAAAGAGGAACGTGACCTCTA
CTCTACGTCGTTGGCGTTCCGACTCCTGCGCGAGCATGGCTTCCAAGTT
GCCCAGGAAGTCTTTGATAGTTTCAAGAATGAGGAAGGCGAGTTTAAAG
AGAGCCTGTCGGACGATACGCGAGGCTTGTTGCAGTTGTACGAGGCTTC
GTTCCTGCTCACGGAAGGAGAAACCACTCTCGAATCTGCCCGCGAGTTT
GCAACGAAATTCTTGGAAGAAAAGGTGAACGAAGGTGGCGTGGATGGAG
ACCTCCTGACTCGTATCGCGTACTCGCTGGATATTCCGTTGCACTGGCG
CATCAAGCGCCCCAATGCCCCGGTCTGGATCGAATGGTATCGTAAACGA
CCAGATATGAACCCGGTTGTTCTCGAGCTGGCTATCCTGGACCTGAATA
TTGTGCAGGCACAATTTCAAGAGGAGTTGAAGGAGTCTTTTCGCTGGTG
GCGCAATACAGGCTTTGTGGAAAAACTGCCATTCGCGCGCGATCGCCTC
GTGGAATGCTACTTCTGGAACACTGGTATTATCGAGCCGCGTCAGCATG
CGTCGGCGCGCATCATGATGGGAAAGGTCAATGCTCTGATCACGGTGAT
CGACGACATCTATGATGTCTACGGTACCCTGGAGGAGCTGGAACAATTC
ACAGATCTGATTCGGCGCTGGGATATTAATAGCATTGATCAGCTGCCTG
ATTACATGCAACTGTGCTTTTTGGCTCTCAATAATTTTGTTGACGATAC
CTCCTATGATGTTATGAAAGAAAAAGGCGTGAATGTTATCCCCTATCTC
CGCCAATCTTGGGTTGACTTGGCAGATAAGTATATGGTTGAGGCGCGGT
GGTTTTACGGTGGGCACAAGCCAAGTCTGGAAGAATATTTGGAGAACAG
TTGGCAGAGTATTTCTGGCCCTTGCATGCTCACGCACATCTTTTTTCGA
GTGACGGACAGTTTTACCAAGGAGACGGTGGATAGCCTGTATAAATATC
ATGATCTCGTCCGTTGGAGCTCTTTCGTGTTGCGCCTGGCAGATGACCT
GGGTACTAGCGTGGAGGAGGTTTCGCGCGGTGACGTGCCTAAAAGCCTG
CAGTGCTATATGAGTGATTACAACGCCTCGGAAGCAGAGGCTCGGAAAC
ACGTCAAATGGCTGATCGCTGAGGTGTGGAAAAAAATGAATGCTGAGCG
CGTGAGTAAGGACAGCCCATTCGGCAAAGATTTTATTGGCTGCGCGGTG
GATCTGGGCCGCATGGCCCAACTGATGTACCACAATGGTGATGGCCACG
GCACACAACACCCAATCATCCATCAACAGATGACCCGGACCCTCTTTGA
ACCTTTCGCATAA

PsbA promoter→BOLD font
Synthetic ribosomal binding site (RBS) continuous promoter→italic font
Codon optimized limonene synthase→plain font

### II. Algal Cultivation Systems (Pools, Ponds and Bioreactors)

Algal antennae are highly efficient at absorbing almost all photons that hit them, leading to mutual shading.¹⁰ The lack of thorough, quantitative understanding of mutual shading hinders light management and hampers algal growth potential. Precise light distribution pattern (LDP) prediction could guide an innovative cultivation design to unleash growth potential. However, most current computational models predict LDPs as one-dimensional light paths that are not representative of real-world LDPs. ^{9, 10, 11, 12, 13, 14} Moreover, these models perform poorly at high cell concentrations with more severe light scattering and diffusive reflection.^{9, 10, 11, 12, 13, 14} Machine learning based on empirical training could overcome these challenges to achieve two- or even three-dimensional LDP predictions.

Besides the growth limitation, high costs and energy demands associated with harvesting and dewatering represent another significant technical barrier³, creating an inherent dilemma between light availability and harvesting cost. High cell concentration is preferred for algal biomass harvesting to minimize cost per unit, but it will inevitably result in strong mutual shading that limits growth. Traditional methods like centrifugation, filtration, chemical flocculation, or bio-flocculation can make up as much as 30% of total costs and 50% of total energy use, which makes them impractical for frequent harvests to bypass mutual shading.^{3, 5, 15, 16} A cost-effective harvesting method is thus urgently needed to address this dilemma.

This study provides a solution for the aforementioned challenges with a cultivation design informed by machine learning and a synthetic biology-based platform implementation.

In general, cultivation conditions (e.g., light intensity and temperature) determine the range of OD for triggering harvesting. However, cyanobacteria will significantly be inhibited by mutual shading such that harvesting is recommended.

Machine learning was demonstrated as an effective LDP-prediction tool to assess light availability inside algal culture.

Light availability was used to predict cyanobacterial growth rates with a second machine learning model, GRM (growth rate prediction model). Together, the machine learning models allow accurate growth simulation and guide the design of a semi-continuous algal cultivation (SAC). SAC sustains optimal growth rates to minimize mutual shading and drastically increases biomass productivity.

A strategy of aggregation-based sedimentation (ABS) achieved low-cost harvesting and cost-effective SAC implementation.

Embodiments, described herein, are used in bodies of water, including but not limited to pools, ponds, bioreactors, etc.

### A. Aggregation-Based Sedimentation (ABS)

ABS is achieved by engineering Synechococcus elongatus UTEX 2973 (UTEX 2973) to produce limonene, which generates hydrophobic surface interaction and triggers cell aggregation for sedimentation. Moreover, the strain co-produces biomass as a potential fuel precursor and limonene as a value-added product. Scaling-up of the machine learning-informed SAC with an outdoor pond system also shows a high biomass productivity. The impacts of high yields from SAC and a simplified harvest method are assessed with a techno-economic analysis (TEA).

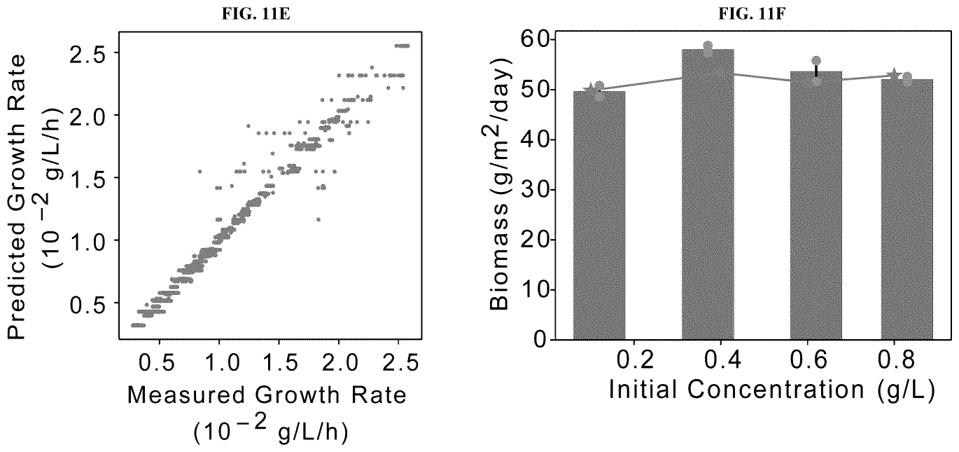

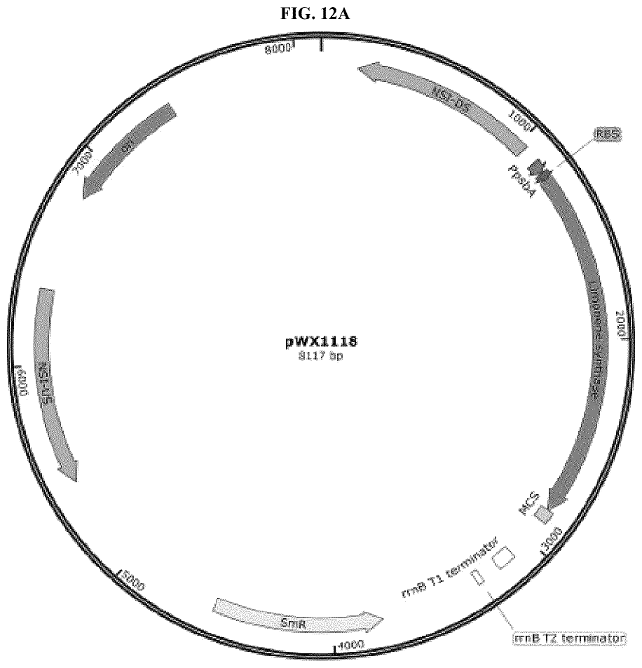

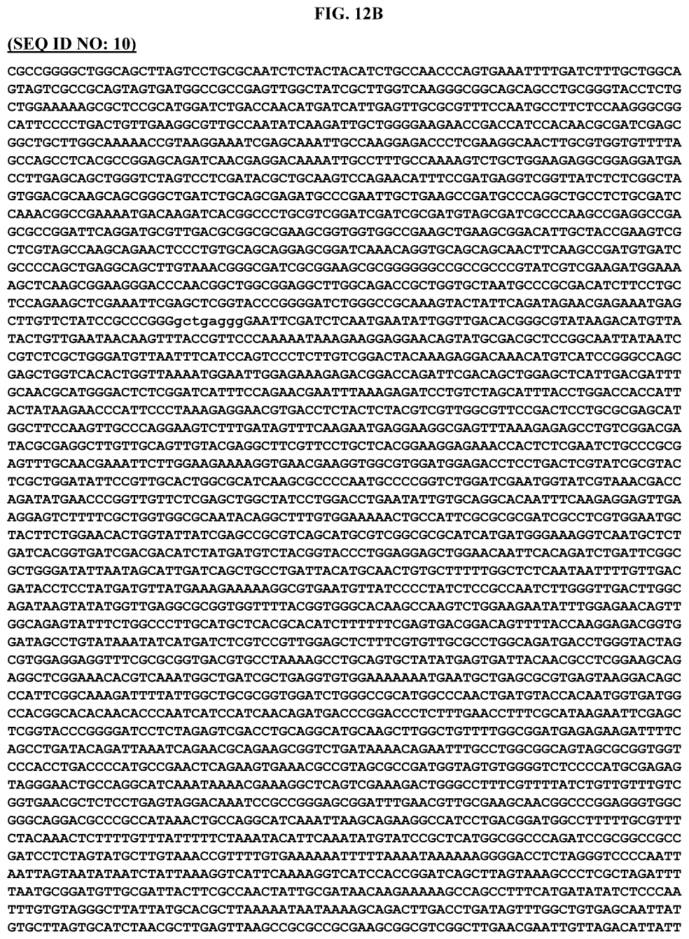

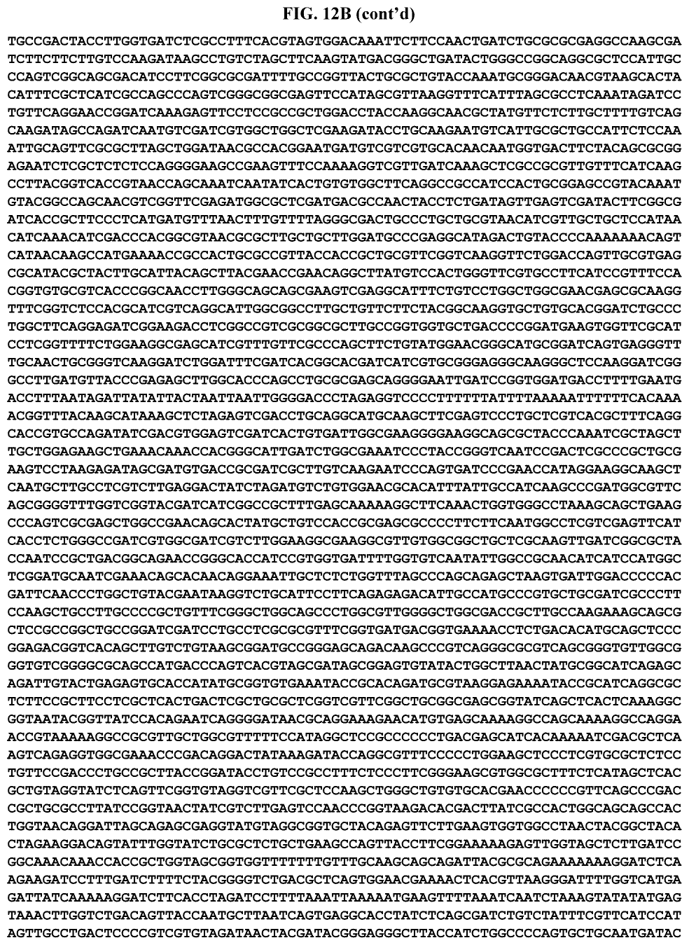

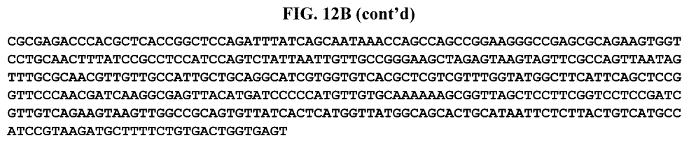

A customized pond system was used for scaling-up of the SAC, shown in FIG. 11A. The circular pond system contained a 6-inch-wide raceway and an impeller was used to keep the cyanobacterial cells agitated. 30 liters of cyanobacteria (20 cm in height) were cultivated in the pond system with 5% CO₂ (vol/vol) bubbling via gas dispersion stones. The growth temperature was maintained at 40° C. with a water heater. Cell growth and light conditions were monitored with a turbidity meter (EXcell231, EXNER, with Expert software) and a light sensor (LS-BTA, Vernier, with Vernier Graphical Analysis software), respectively. In the condition mimicking Texas summer, the pond system was placed in a growth chamber and the light program was set to 400 μmol m⁻² s⁻¹ for 1 h, 800 μmol m⁻² s⁻¹ for 1 h, 1300 μmol m⁻² s⁻¹ for 1 h, 1500 μmol m² s⁻¹ for 10 h, 1300 μmol m⁻² s⁻¹ for 1 h, 800 μmol m⁻² s⁻¹ for 1 h, and 400 μmol m⁻² s⁻¹ for 1 h (all light intensities were measured from the pond surface). In both outdoor and mimicking outdoor condition, 250 ml water was added to the pond system every 2 hours to counter evaporation.

### B. SAC Pond Systems

SAC was validated with a 30-liter raceway pond system. Machine learning models (LDPM and GRM) were adapted for a pond system to guide the cultivation design. Both models showed high prediction accuracy. The LDPM achieved an overall R² score of 0.986 (FIG. 11B) and pixel-by-pixel analysis suggested the LDP prediction was reasonably good at all pixels, with a minimal R² score of 0.943 (FIG. 11C). The GRM prediction also achieved an R² score of 0.980 (FIG. 11E). Like the photobioreactor (PBR) system, we employed the machine learning models to predict optimal initial cell concentrations for the pond SAC system. The growth simulation suggested that setting initial cell concentration to around 0.4 g/L delivers the highest biomass productivity under the growth condition mimicking Texas summer (FIG. 11F). Based on the prediction, the experimental results showed that SAC achieved the highest biomass productivity at 58.1 g/m²/d (FIG. 11F). We noticed slight differences between the predicted biomass productivity and measured productivity when initial cell concentrations were around 0.4 g/L (FIG. 11F). The deviation might result from the presence of noise in the training data, and/or overfitting in the models. Future optimization such as removing noise, adding regulations, and expanding training data could further enhance the model performance. Overall, our results demonstrated the application of machine learning models in a pond SAC system. The success of application in both PBR and pond systems indicates that machine learning-based prediction can be a generalized method for guiding algal cultivation management and design in various systems.

Inspired by the high productivity from the indoor pond system, we further tested biomass productivity of the pond SAC in real outdoor conditions. The outdoor tests were carried out in late September 2021 in College Station, Texas, with both ‘partially sunny’ and ‘mostly sunny’ weather. These conditions represent a typical fall growth condition. The outdoor cultivation achieved an average biomass productivity of 43.3 g/m²/d (FIG. 11D), surpassing the U.S. DOE 2022 target by 1.7 times.

### C. Pond SAC System Techno-Economic Analysis

The machine learning-informed SAC holds significant economic potential after being scaled up. Recent efforts to quantify the economic potential of algal biomass production by the National

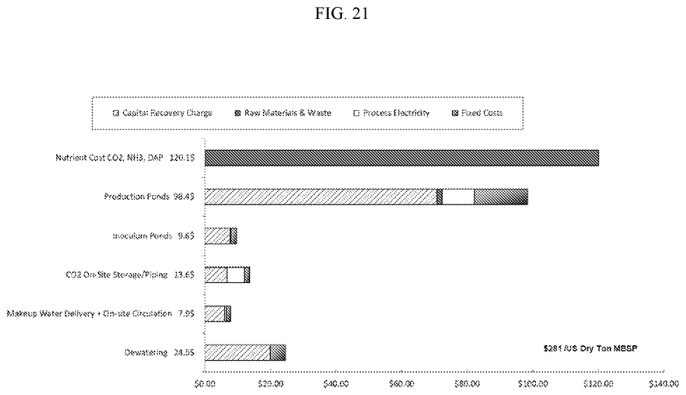

Renewable Energy Laboratory (NREL) examined different existing, well-documented PBR and pond designs across a number of different configurations.^{44, 45} Both studies focused on estimating the break-even minimum biomass selling price (MBSP), given an internal rate of return on capital of 10%. Based on the NREL study, the yearly average of biomass productivity is estimated to be the productivities achieved in the Spring (March, April, May) and Fall (SEP, October, November).⁴⁴ Following that approach, we estimated the yearly average of biomass productivity for the open pond system to be 43.3 g/m²/d in the outdoor study and 48.1 g/m²/d (83.3% of summer productivity) in the indoor mimicking trial. The ash content of the cyanobacterial biomass was measured to be 5.5%. At these conditions, the NREL model projects a MBSP of approximately $281 per ton based on the outdoor trial yield. By comparison, 2019 state-of-the-art open pond algal cultivation had an MBSP of approximately $1,227 per ton.⁴⁶ See, FIG. 21.

Furthermore, the limonene produced by L524 has a current market value of about $5/kg.^{29, 40} At this price, the SAC system proposed here would generate approximately $10.08 of additional revenue in limonene sales per ton of biomass produced. Such reductions in MBSP can be readily achieved in PBR systems. Although limonene collection from open pond systems may not be cost effective at current productivity levels, limonene-mediated ABS nonetheless significantly reduces harvesting costs.

Beyond significant improvements in biomass production, the implementation of ABS in SAC would also markedly reduce operating costs. ABS (0.1 kWh m⁻³) could save up to 93% on energy costs compared to traditional harvesting methods (e.g., disc stack centrifugation (1.4 kWh m⁻³)),³ while maintaining high efficiency and recovery rates. As the dewatering process accounts for $24.4 per ton of biomass in the current model, the simplified harvest by ABS would further significantly reduce the MBSP (however, we have not adjusted the $281 per ton MBSP generated by the NREL model to reflect such reductions). See, FIG. 21.

In addition, due to the high glycogen content of UTEX 2973 cells⁴², the cyanobacterial biomass could directly feed into biorefineries for ethanol fermentation without pretreatment as described previously.^{47, 48} Demand for biomass is not considered by the NREL model used here, so the additional benefit of increased willingness-to-pay for biomass from the L524 and SAC platform is not quantified. While still in the early stages of development, the SAC platform with the L524 strain appears to overcome many of the challenges that have long plagued algal biofuel production.

Together, significant increases in algal productivity and reductions in operating costs result in a dramatic reduction in the break-even biomass price relative to prior algal production systems to below $300 per ton of AFDW. Detailed work must be done to provide robust cost estimates, but the initial results show great promise. At the same time, the SAC process would generate biomass that is significantly less costly to convert to ethanol than the current most common feedstock (corn), as it would eliminate the need for costly milling and other pre-treatment prior to fermentation.^{47, 48}

### III. Machine Learning Models

Machine learning models allows for accurate prediction of growth simulation and as a guide the design of a semi-continuous algal cultivation (SAC).

### A. Light Availability Prediction (LDP)

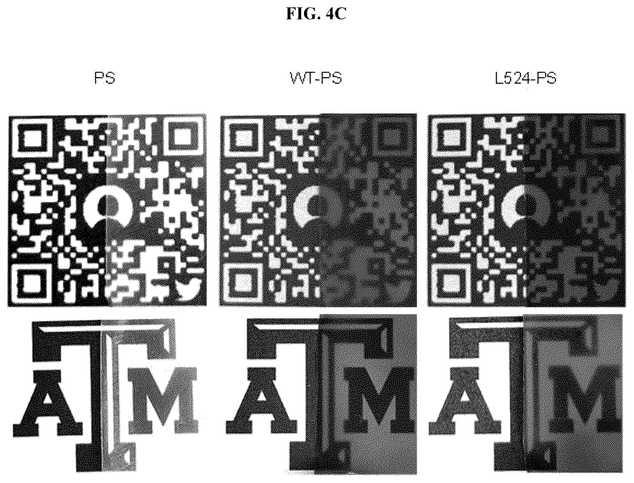

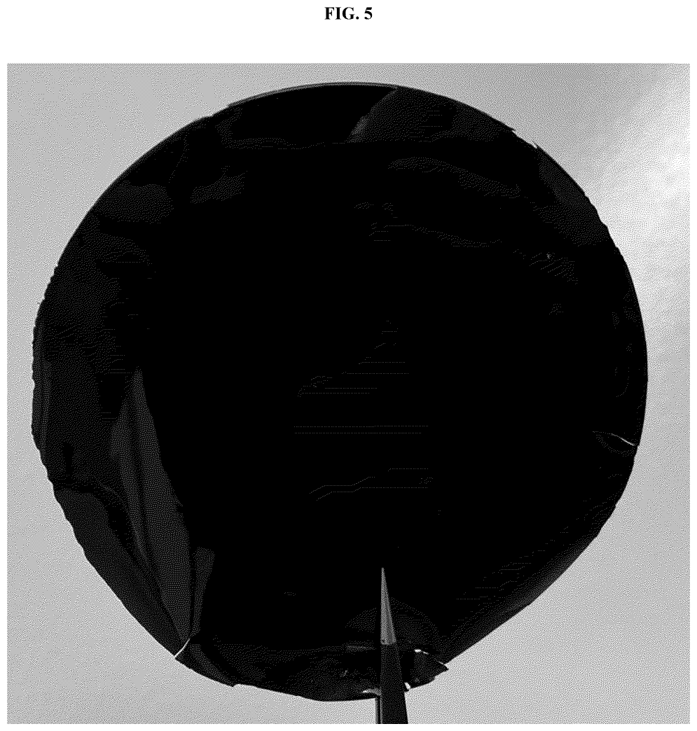

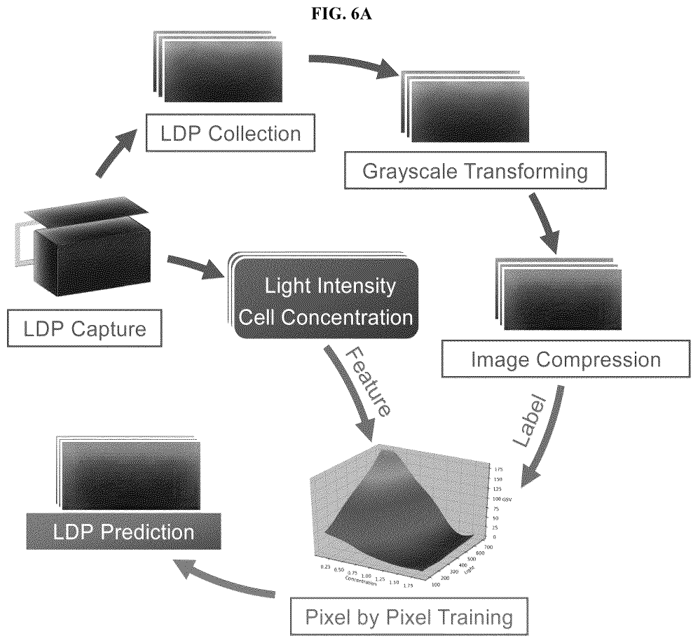

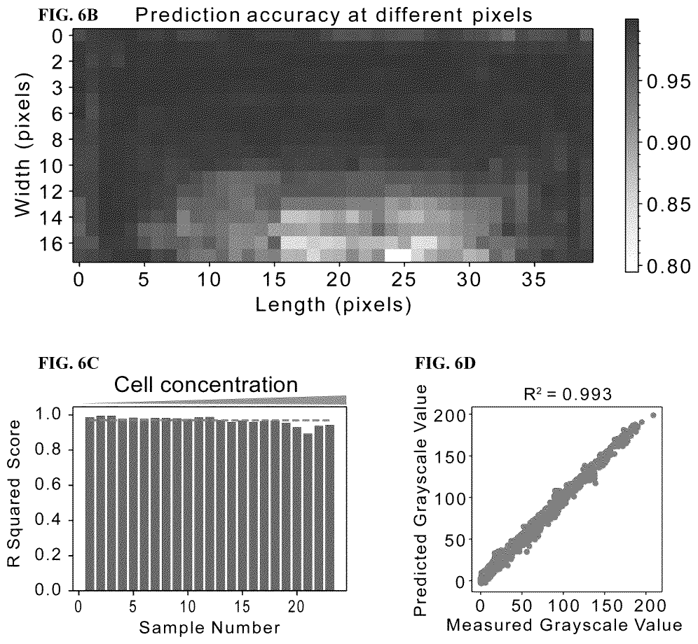

Building machine learning models for LDP prediction considers the asymmetry of light sources in most PBRs and raceway ponds. LDPs may be two-dimensional or three-dimensional. Here, a two-dimensional grayscale image was developed to represent an LDP, with grayscale values (GSV, range of 0 to 255 with 0 for black and 255 for white) representing light intensities. See, Example II The GSVs and light intensities showed a strong linear correlation with an average R² score of 0.969 across a wide range of cell concentrations, validating the approach (FIG. 6C). Next, we evaluated the effectiveness of machine learning in LDP prediction. The overall workflows of sample preparation and training processes are shown in FIG. 6A. Light intensity and cell concentration, the two major factors determining LDPs, were set as features and their corresponding LDPs were set as labels in training. We chose the Support Vector Regression (SVR) algorithm to train due to its versatility,^{17, 18, 19} resulting in an LDP prediction model (LDPM, See, Example III).

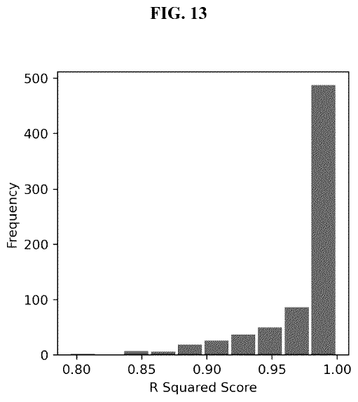

Evaluation of the LDPM prediction showed an R² score of 0.993 between all predicted LDPs and measured LDPs (FIG. 6D), indicating high prediction accuracy. A pixel-by-pixel evaluation of the entire LDP suggested that 94.4% of pixels achieved R² values higher than 0.90, and 0.8% of pixels had R² values in the range of 0.79 to 0.85 (FIG. 6B, FIG. 13., indicating precise predictions at most pixels. Pixels further away from the light source (row 12-row 18) showed relatively lower R² scores (FIG. 6B), presumably because of the increased complexity of the light pattern. Overall, the accurate LDP prediction proves the feasibility of using machine learning to model light availability inside algal cultures. The high R² score (0.993) highlights the increased accuracy of the machine learning model over traditional mathematical models.^{10, 13, 14} Furthermore, unlike mathematical models that can predict one-dimensional light paths, machine learning-predicted LDPs can be two-dimensional or even three dimensional. Moreover, the upper cell concentration limit of the LDPM is about 3.9 g/L, which is higher than the limit of ˜1 g/L presented in previous mathematical models^{10, 13, 14} The larger prediction range indicates that a machine learning-based strategy could address LDP prediction challenges caused by complex light scattering and interference at high cell concentrations. The methodology for LDP prediction proposed in this study could be transferred to any existing algal cultivation systems, such as indoor/outdoor PBRs or pond systems. The superior performance of the machine learning model—in particular, a larger prediction range and higher accuracy-enabled LDP outputs to be used to simulate growth curves using a second machine learning model. Such integration has not been achieved in previous studies and would guide cultivation optimization.

### B. LDP Growth Rate Prediction

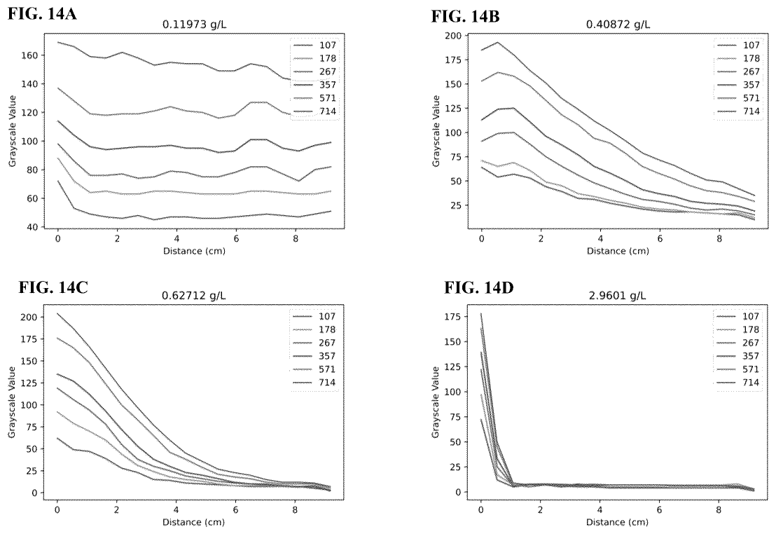

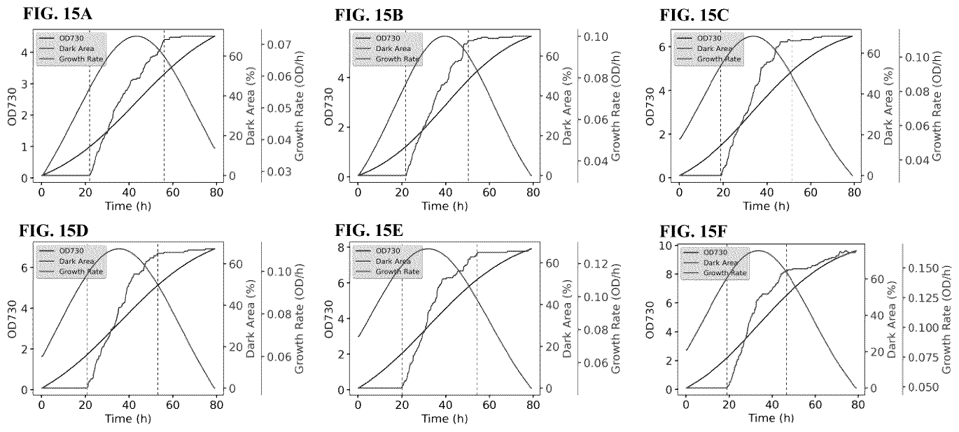

The LDP prediction allowed us to quantify mutual shading and explore the impact of light availability on cyanobacterial growth. It was found that the shading effect increased sharply when cells grew to a high concentration, similar to previous studies^{13, 20}. See, FIG. 14. Cyanobacterial growth rates peaked when dark areas, defined as pixels with GSVs less than 25.5 (10% of the maximal value), and reached 43.1±4.9% at all tested light conditions. See, Example IV. The growth rate dropped drastically when dark areas reached a plateau around 65% See, FIG. 15. Specifically, when dark areas reached 43.1%, cell growth began to be inhibited by mutual shading. Such inhibition intensified after dark areas reached 65%. The strong correlation between light pattern and growth rates suggests that light availability is the primary factor determining cyanobacterial growth rates when nutrients are sufficient and temperature is controlled. The results are consistent with previous findings that light availability defines the growth potential for cyanobacteria given abundant nutrients.^{21, 22} Quantitative understanding allowed us to develop a second machine learning model to predict growth rate based on LDPs. We named this second machine learning model a growth rate prediction model (GRM).

### C. Growth Rate Prediction Model (GRM)

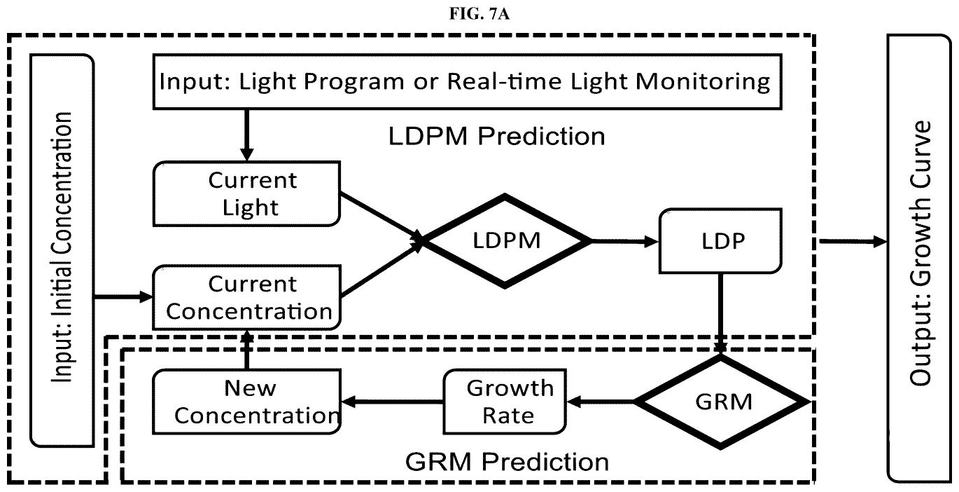

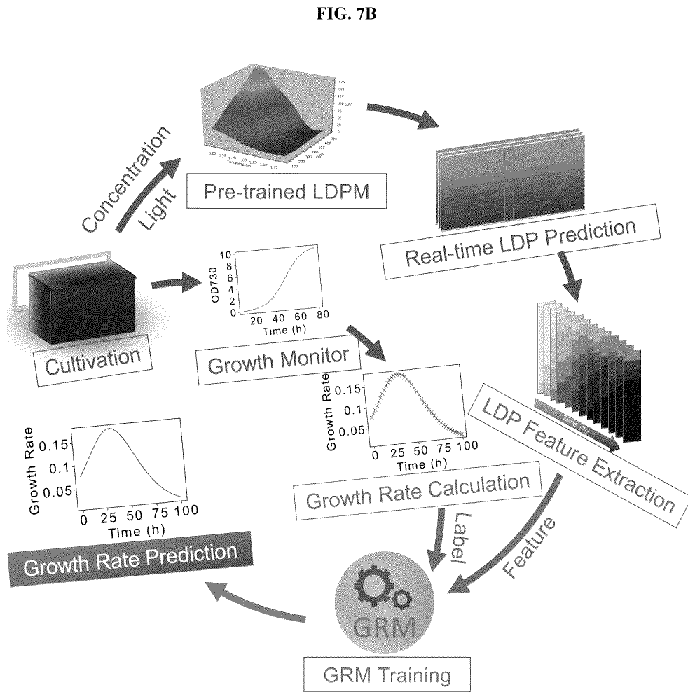

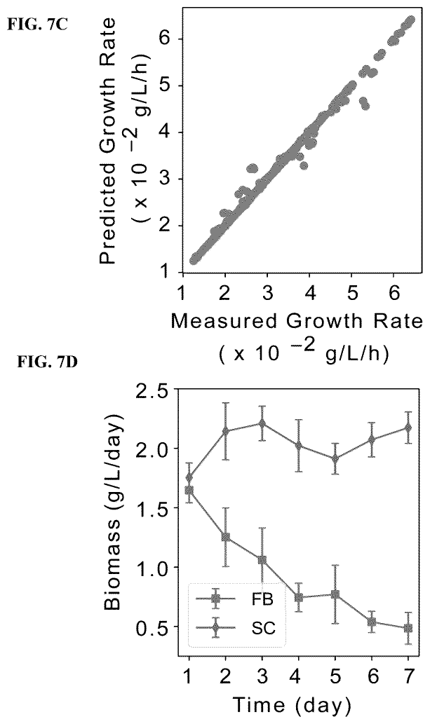

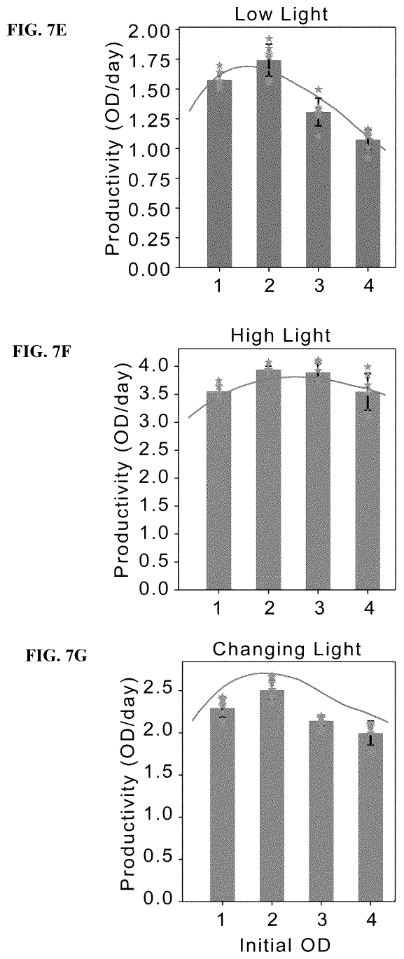

A method was developed for predicting cyanobacterial growth rates with a growth rate prediction machine learning model (GRM). See, FIG. 7B. Vectors extracted from LDPs and their corresponding growth rates (based on the same time points) were set as features and labels in the training, respectively. See, Examples V and VI. Validation rendered an R² value of 0.992, verifying the accuracy of GRM prediction. See, FIG. 7C. The results established quantitative connection between light availability and cell growth rates. The success in growth prediction indicates that machine learning could be introduced as an effective tool to monitor or simulate algal growth, inform light management, and guide cultivation system design.

### D. Semi-Continuous Algal Cultivation (SAC)

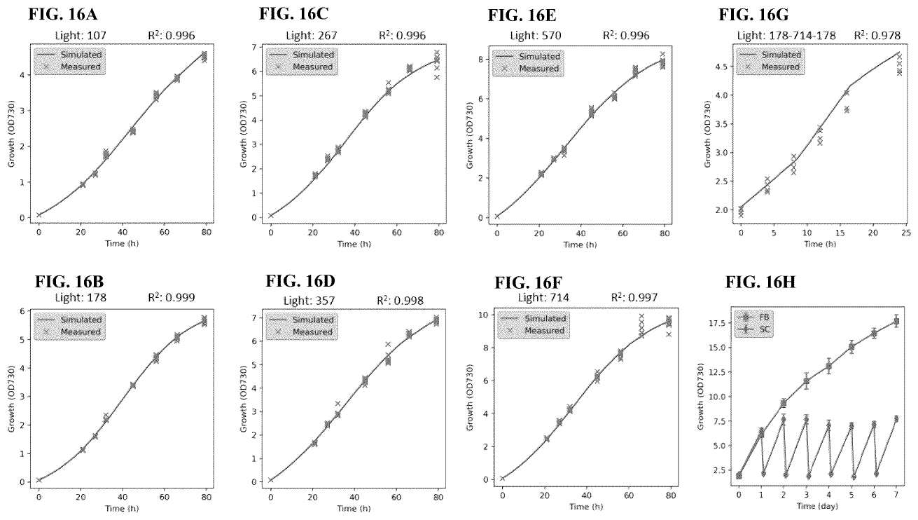

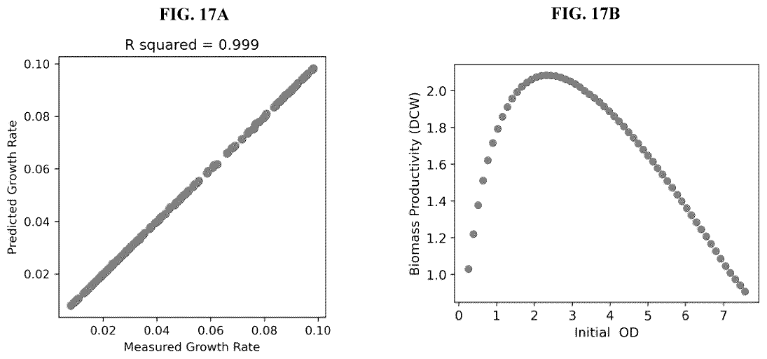

The ability to predict algal growth contributes to efficient algal cultivation management and design. For example, given light conditions over the coming days and current cell concentrations, growth prediction could indicate the optimal harvest time and how much to harvest for maximum productivity and profit. Machine learning models resulted in systems that simulate cyanobacterial growth under different constant light conditions by combining the LDPM and GRM. See, FIG. 7A, and Example VII. The simulated growth was very close to measured growth at all tested conditions, with a lowest R² value of 0.996. See, FIG. 16A-H. Machine learning models were tested for simulation of cyanobacterial growth under changing light conditions. Growth predictions under changing light achieved an R² score of 0.978 compared to measured results, validating the accuracy of the model. See, FIG. 16G.

Overall, the results demonstrated that machine learning models could accurately simulate cyanobacterial cell growth at both constant and changing light conditions. The machine learning model is thus more versatile compared to traditional mathematical models (e.g., models based on the Monod equation) and does not require prior knowledge of growth characteristics. Moreover, the machine learning-based growth simulation is highly flexible and could expand to integrate other growth impacting factors such as temperature and nutrients. Such integration might be too complicated for traditional mathematical models, especially under changing light.

The data presented herein show that machine learned growth simulation models result in cyanobacterial cultivation that overcomes disadvantages of mutual shading. Although many strategies (e.g., illumination optimization, increasing bubbling rates) have been proposed to overcome light limitation, their productivity improvements were limited and not sustainable.^{21, 23, 24}

Empowered by growth prediction, algal cultivation systems where cells are removed periodically or continuously to maintain the cultivation with near-optimal light availability and growth rates were developed. Continuous or semi-continuous cultivation systems minimize the impact of mutual shading and improve growth potential for cyanobacteria. For example, a SAC system was tested with a harvesting (e.g., removing cyanobacteria) interval of 24 hours, (although shorter or longer intervals can be used), and machine learning-based growth simulations that predict optimal initial inoculum concentration.

Biomass productivity predictions were obtained at different initial cell concentrations under low light (107 μmol m⁻² s⁻¹), high light (714 μmol m⁻² s⁻¹), and changing light (178-714-178 μmol m⁻² s⁻¹). As shown, the simulated productivities showed similar trends to measured productivities at all tested light conditions. Measured productivities from constant light conditions were very close to predicted productivities, while minor deviations were observed under changing light. The deviation could have resulted from slower growth due to adaptation to light changes. FIG. 7E-G

Overall, the results reveal the effectiveness of machine learning-based growth simulation in guiding cultivation platform advancement. In real world applications, in addition to predicting optimal initial cell concentration, growth simulation could determine when and how much algal biomass to harvest under certain growth conditions. The prediction could be used in combination with economic analysis for maximized profits.

Despite higher biomass productivity using optimal initial cell concentrations in SAC, the growth rate of UTEX 2973 was less than previously reported.^{25, 26, 27} In order to further improve biomass productivity, we optimized light conditions with double light sources at 574 μmol m⁻² s⁻¹ on opposite sides of PBRs. To determine the best initial cell concentration for the updated SAC, we adapted the machine learning models for double-light growth simulation. The prediction suggested that OD₇₃₀ around 2.3 is the optimal initial cell concentration for SAC. Thereafter, a SAC system was tested under double light sources at 574 μmol m⁻² s⁻¹ and maintained an approximate initial 2.3 OD₇₃₀ after each harvest to allow cell cultivation from an optimal starting concentration. Cyanobacterial biomass productivities in SAC were evaluated with fed-batch cultivation (FB) as a control. The growth of cyanobacteria in fed-batch and SAC is shown in FIG. 4h. Biomass productivities in SAC were maintained at ˜2.0 g/L/day over seven days, while productivity in fed-batch cultivation decreased to 0.4 g/L/day on day 7. FIG. 7D.

The results suggest that machine learning-informed SAC effectively overcomes growth limitations caused by mutual shading and significantly improves and sustains biomass productivity. Such success could encourage further development in artificial intelligence to guide algal cultivation system design, refine cultivation management, and automate process operation.

### E. Aggregation-Based Sedimentation (ABS) Coupled Semi-Continuous Algal Cultivation (SAC)

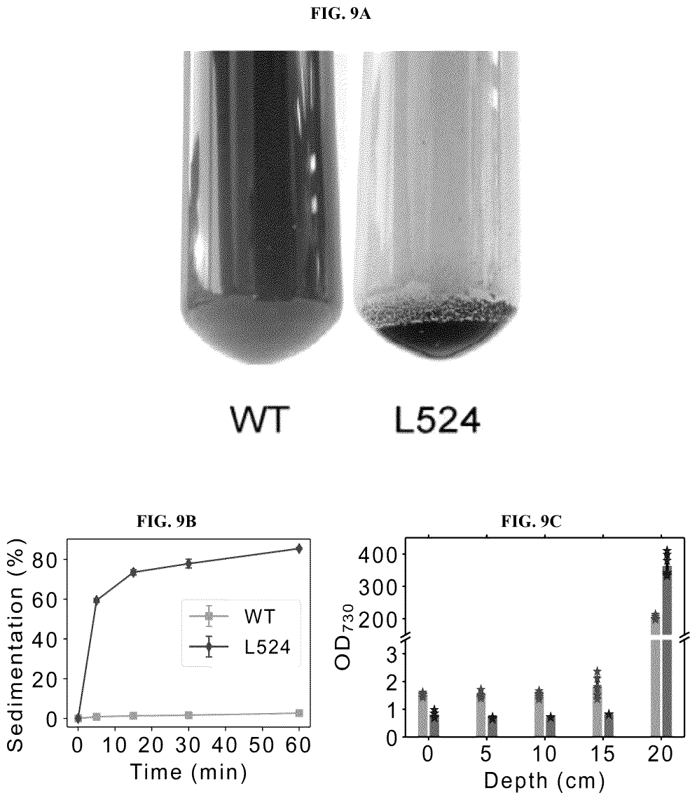

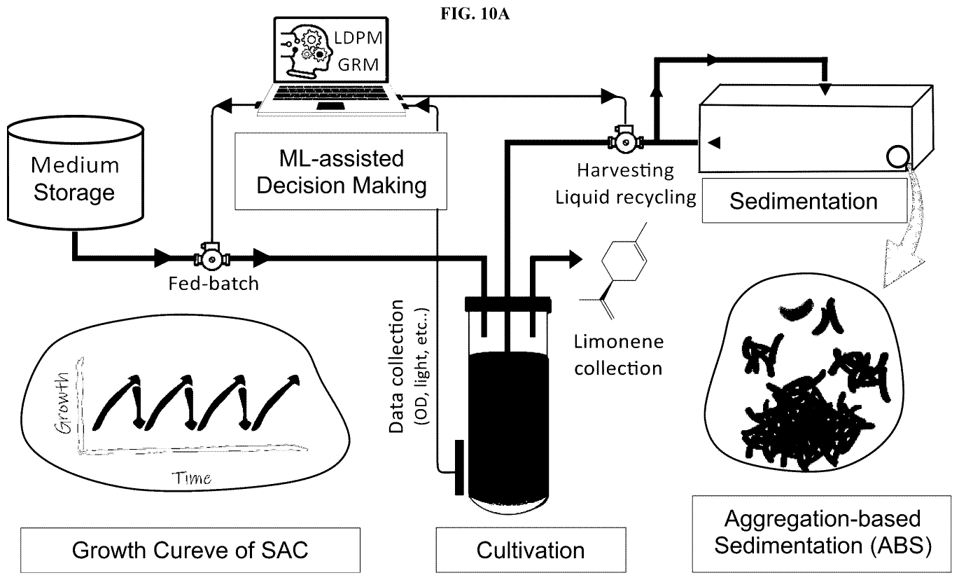

To investigate if limonene-induced aggregation could enable efficient UTEX 2973 cell sedimentation an Aggregation-Based Sedimentation (ABS) process of L524 cells was monitored. (FIG. 9A-B). ABS started within 5 minutes in L524, with over 75% of cells settled after 15 minutes (FIG. 9B). Moreover, 85% and 93% of cells settled to the bottom of the collecting vessel (20 cm in depth) within 0.5 and 6 hours, respectively (FIG. 9C). The results highlight the high recovery rate and settling velocity of ABS. A major disadvantage of algal sedimentation or auto-flocculation is the low solids concentration of the output, typically between 0.5% and 3%³. In contrast, the cell concentration in ABS outputs reached 139.2 g/L, leading to about 14% solids content. The high solid content could result from the hydrophobic effects of limonene. No significant differences were found between the growth of the wild-type and L524, suggesting that the limonene-induced ABS is physically prevented by air/CO₂ bubbling during cultivation. See, FIG. 19.

Overall, we demonstrated a harvest method through manipulating cell surface hydrophobicity. ABS is a cost-effective strategy with high recovery rates, sedimentation velocity, and solid content in the output. ABS could enable a sustainable and cost-effective SAC.

### F. Sustainable ABS/SAC Systems

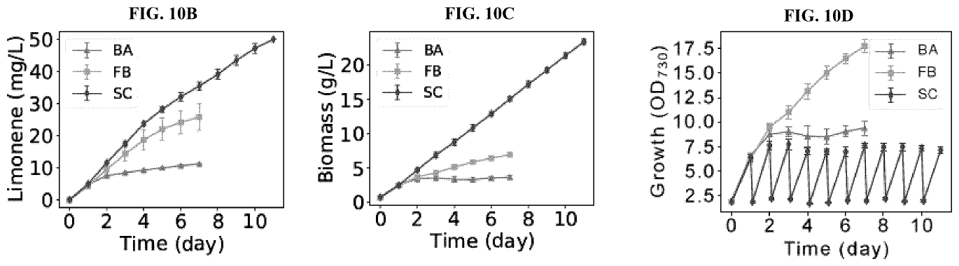

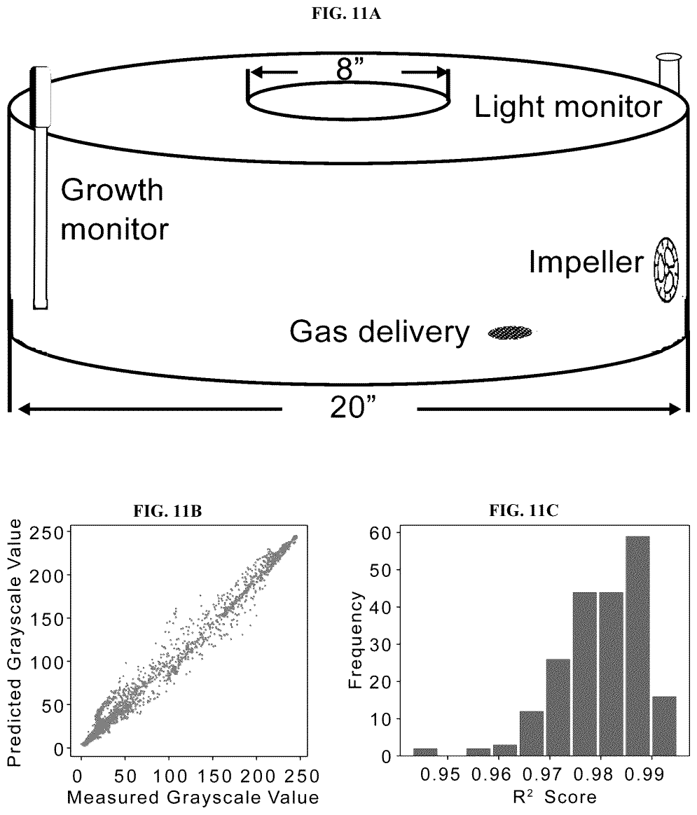

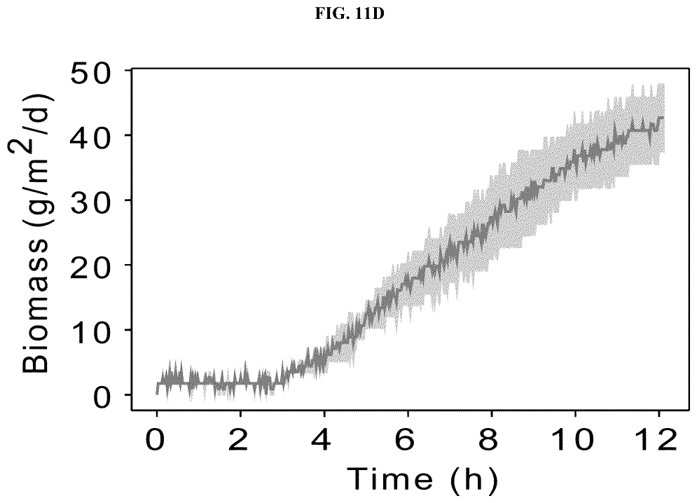

Machine learning-informed SAC and ABS can be integrated for sustainable biofuel production. FIG. 10A. Besides triggering ABS for cost-effective SAC, limonene could also serve as a secondary bioproduct due to its high value and potential application in fragrance, food, and pharmaceutical industries.^{37, 38, 39} Moreover, due to its high energy density, limonene has been regarded as a ‘drop-in’ fuel amenable to aviation and diesel, biodiesel, applications.^{29, 40, 41} Thus, L524 could co-produce limonene and glycogen-rich biomass⁴² from SAC.

L524 cell SAC limonene and biomass production and yield was compared to batch and fed-batch cultivations. In batch cultivation, L524 produced 11.2 mg/L limonene and 3.7 g/L biomass in 7 days. (FIG. 10B-C). The limonene and biomass production was drastically slowed after day 2, indicating growth limitations causing by nutrient depletion (FIG. 10B-C). The limonene and biomass yields increased to 25.8 mg/L and 6.9 g/L, respectively, in 7 days with fed-batch cultivation, which removed the nutrient limitation (FIG. 10B-C). Despite the significant increases, limonene and biomass productivities still gradually decreased over time, suggesting that mutual shading became a limiting factor at high cell concentration (FIG. 10B-D). In contrast, by overcoming mutual shading, the SAC sustained near-linear limonene and biomass accumulations of about 5 mg/L/day of limonene and 2.2 g/L/day of biomass (FIG. 10B-C). The sustained high productivity resulted in 50.0 mg/L of limonene and 23.4 g/L of biomass over 11 days (FIG. 10B-C).

### IV. Bioremediation

Microplastics (MPs) and nanoplastics (NPs) have been linked to various health problems in living systems. Bhatt et al., 2021. “Microplastic contaminants in the aqueous environment, fate, toxicity consequences, and remediation strategies” Environmental Research 200. Because of their small size, microplastics are hard to clean and easy to enter the food chain through the consumption of aquatic food products. Pico et al., 2019. “Nano- and microplastic analysis: Focus on their occurrence in freshwater ecosystems and remediation technologies” Trac-Trends in Analytical Chemistry 113:409-425.

One possible solution to this problem is bioremediation, which involves using microorganisms to remove MPs and NPs. In recent years, many biological strategies have been proposed and developed to remediate different types of plastics. Hu et al., 2021. “Microplastics remediation in aqueous systems: Strategies and technologies” Water Research 198. While a major focus of MP bioremediation is using microorganisms or microbe-derived enzymes to degrade plastic polymers, their degradation efficiency in the nature environment might not be optimal. Tournier et al., 2020. “An engineered PET depolymerase to break down and recycle plastic bottles. Nature 580:216-+; Sanchez C., 2020. “Fungal potential for the degradation of petroleum-based polymers: An overview of macro- and microplastics biodegradation” Biotechnology Advances 40; Jacquin et al., 2019. “Microbial Ecotoxicology of Marine Plastic Debris: A Review on Colonization and Biodegradation by the “Plastisphere” Frontiers in Microbiology 10; and Yoshida et al., 2016. “A bacterium that degrades and assimilates poly(ethylene terephthalate). Science 351:1196-1199.

Moreover, direct plastic depolymerization in the environment may release toxic substances, highlighting the need of capture MPs first and then conducting MP degradation and/or upcycling in a separated and closed space. Until today, very limited studies have been reported in microbe-based MP captures, most of which took advantages of the natural aggregation and biofilm formation properties of microorganisms. See, Table 4; de Oliveira et al., 2020. “Interaction of Cyanobacteria with Nanometer and Micron Sized Polystyrene Particles in Marine and Fresh Water’ Langmuir 36:3963-3969; Cunha et al., 2020. “Microalgal-based biopolymer for nano- and microplastic removal: a possible biosolution for wastewater treatment” Environmental Pollution 263; and Cunha et al., 2019. “Marine vs freshwater microalgae exopolymers as biosolutions to microplastics pollution” Environmental Pollution 249:372-380.

TABLE 4
Summary of microorganism-based MP capture.

| | | | | Removal | Characterization | |
|---|---|---|---|---|---|---|
| Species | Interaction | Design | MP types | efficiency | method | Refs |
| Microcystis | EPS- | N.A. | PS and PMMA | N.A. | Imaging | (41) |
| panniformis | mediated | | (<106 μm and | | (photograph, | |
| Scenedesmus sp. | aggregates | | 106-250 μm) | | micrograph & SEM) | |
| Tetraselmis sp. | | | | | | |
| Gloeocapsa sp. | | | | | | |
| Cyanothece sp. | EPS- | N.A. | PS (0.1 and | N.A. | Imaging | (40) |
| | mediated | | 10 μm) | | (photograph and | |
| | aggregate | | | | micrograph) | |
| Synechococcus | EPS- | N.A. | PS (0.1 and | ~18% | Turbidity and | (39) |
| elongatus PCC | mediated | | 10 μm) | normalized | Imaging | |
| 7942 | aggregate | | | OD reduction | (photograph, | |
| | | | | in 6.5 h | micrograph, | |
| Synechococcus | | | | ~82% | and SEM) | |
| sp. PCC 7002 | | | | normalized | | |
| | | | | OD reduction | | |
| | | | | in 6.5 h | | |
| Pseudomonas | EPS- | Plasmid | PET, PMMA, | N.A. | Raman | (43) |
| aeruginosa | mediated | ΔwspF/ | nylon 6/6, and | | Microspectroscopy | |
| | aggregate | pBAD- | PVC (<106 μm); | | and Imaging | |
| | | yhjh | MPs from | | (photograph, | |
| | | | seawater samples | | micrograph & SEM) | |
| | | | (106-300 μm) | | | |
| Pseudomonas | EPS- | MP | PS, PET, and | N.A. | Raman | (42) |
| aeruginosa | mediated | aggregate | PMMA (<106 μm); | | Microspectroscopy | |
| | aggregate | lab | MPs from | | and Imaging | |
| | | evolution/ | seawater samples | | (micrograph) | |
| | | trypsin | (106-300 μm) | | | |
| Synechococcus | Hydro- | Over- | PS (0.2-5 μm), | Depends on | Turbidity | This |
| sp. UTEX 2973 | phobic | expressed | PET (<300 μm), | MP types | Raman | study |
| | aggregate | limonene | and PE (32-38 | | microspectroscopy, | |
| | | synthase | μm); | | Imaging | |
| | | | | | (photograph, | |
| | | | | | micrograph, SEM, | |
| | | | | | TEM) & TGA) | |

Alternatively, MP capture and release designs have been reported by controlling the c-di-GMP and biofilm formation in Pseudomonas aeruginosa. Chan et al., 2022. “Microbial-Enzymatic Combinatorial Approach to Capture and Release Microplastics” Environmental Science & Technology Letters doi:10.1021/acs.estlett.2c00558; and Liu et al., 2021. “Engineering a microbial ‘trap and release’ mechanism for microplastics removal” Chemical Engineering Journal 404.

### A. Plastics: Environmental Impact

Due to the low costs, light weight, and high performance of plastics, its production has experienced an explosive increase since the 1950s, jumping from 1.5 million tons in 1950 to 367 million tons in 2020. Europe P. 2021. Plastics—the Facts 2021, p 12, plasticseurope.org/knowledge-hub/plastics-the-facts-2021/. The accumulative plastic production is projected to be 25 billion tons in 2050, with the majority of them unrecycled. Geyer et al., 2017. “Production, use, and fate of all plastics ever made” Science Advances 3. Plastics are highly persistent materials and can last for hundreds of years while breaking down into small pieces, generating microplastics (MPs) and nanoplastics (NPs). Alimi et al., 2018. “Microplastics and Nanoplastics in Aquatic Environments: Aggregation, Deposition, and Enhanced Contaminant Transport” Environmental Science & Technology 52:1704-1724; Dawson et al., 2018. “Turning microplastics into nanoplastics through digestive fragmentation by Antarctic krill.” Nature Communications 9; and do Sul Jai et al., 2014. “The present and future of microplastic pollution in the marine environment” Environmental Pollution 185:352-364.

A standard for MP and NP classification is yet to be established. For example, plastics with diameter of <100 nm were defined as NPs and those with a diameter of 100 nm-5 mm were defined as MPs. Nguyen et al., 2019. “Separation and Analysis of Microplastics and Nanoplastics in Complex Environmental Samples” Accounts of Chemical Research 52:858-866. Plastic particles discussed herein are referred to as microplastics having diameters/dimensions of between 100 nm and 5 mm.

MPs and NPs have been regarded as emerging threats to terrestrial and aquatic ecosystems. Machado et al., 2018. “Microplastics as an emerging threat to terrestrial ecosystems” Global Change Biology 24:1405-1416; and Kogel et al., 2020. “Micro- and nanoplastic toxicity on aquatic life: Determining factors” Sci Total Environ 709:136050. Studies have revealed negative impacts of MPs and NPs on a broad spectrum of living organisms such as microorganisms, plants, and fishes. Miyazaki et al., 2015. “Cytotoxicity and behavior of polystyrene latex nanoparticles to budding yeast” Colloids and Surfaces a-Physicochemical and Engineering Aspects 469:287-293; Nomura et al., 2016. “Cytotoxicity and colloidal behavior of polystyrene latex nanoparticles toward filamentous fungi in isotonic solutions” Chemosphere 149:84-90; Sun et al., 2020. “Differentially charged nanoplastics demonstrate distinct accumulation in Arabidopsis thalia” Nat Nanotechnol 15:755-760; and Wang et al., 2020. “Bioavailability and toxicity of microplastics to fish species: A review” Ecotoxicology and Environmental Safety 189.

More importantly, studies have identified the presence of MPs in a variety of human organs or tissues, including colon, lung, liver, blood and placenta. Ibrahim et al., 2021. “Detection of microplastics in human colectomy specimens” Jgh Open 5:116-121; Amato-Lourenco et al., 2021. “Presence of airborne microplastics in human lung tissue” Journal of Hazardous Materials 416; Horvatits et al., 2022. “Microplastics detected in cirrhotic liver tissue” EBioMedicine 82:104147; Leslie et al., 2022. “Discovery and quantification of plastic particle pollution in human blood” Environment International 163; and Braun et al., 2021. “Detection of Microplastic in Human Placenta and Meconium in a Clinical Setting” Pharmaceutics 13.

Although the exact impacts of MPs on human health are still not conclusive, a recent study revealed a correlation between inflammatory bowel disease and microplastics in feces. Yan et al., 2022. “Analysis of Microplastics in Human Feces Reveals a Correlation between Fecal Microplastics and Inflammatory Bowel Disease Status” Environmental Science & Technology 56:414-421. These studies raised the concerns about potential health risks from MPs, highlighting the necessary of developing low-cost, efficient, and eco-friendly MP remediation systems.

Water is considered as one of major reservoirs and transportation vectors for MPs and NPs. MPs and NPs can be sourced from direct use of products containing plastic particles (e.g., cleansers and cosmetics) and breaking down from plastic materials in the environment. Pico et al., 2019. “Nano- and microplastic analysis: Focus on their occurrence in freshwater ecosystems and remediation technologies” Trac-Trends in Analytical Chemistry 113:409-425. In both cases, a large portion of plastic particles will eventually be carried to aquatic environment, where long-range transportation happens. In addition, many microplastics are not biodegradable, meaning that they can persist for a long time once enter the environment. Bhatt et al., 2021. “Microplastic contaminants in the aqueous environment, fate, toxicity consequences, and remediation strategies” Environmental Research 200. More importantly, transferring into aquatic systems is a major intermediate step for MPs and NPs entering food web, which potentially put threats to human health. Guilhermino et al., 2018. “Uptake and effects of the antimicrobial florfenicol, microplastics and their mixtures on freshwater exotic invasive bivalve Corbicula fluminea” Science of the Total Environment 622:1131-1142; and Silva-Cavalcanti et al. 2017. “Microplastics ingestion by a common tropical freshwater fishing resource” Environmental Pollution 221:218-226.

Thus, MP removal becomes a logical step in the water treatment to reduce the total environmental MPs. The MP removal capacities of current wastewater treatment systems vary from plant to plant, with removal efficiency ranging from 25% to 99.9%. Sun et al., 2019. “Microplastics in wastewater treatment plants: Detection, occurrence and removal” Water Research 152:21-37. Although some systems showed decent MP removal efficiencies, a major drawback of these systems is the lack of downstream infrastructure for processing removed MPs in the sludge. Therefore, the removed MPs could be possibly released into environment again if the sludge is not properly disposed.

Microorganism-based bioremediation represents a promising way for MP removal and subsequent MP processing. One of its advantages is the flexibility and programmability. A wide range of biological modules can be installed into host cells to program microbes for various functionality. For example, microorganisms can be potentially modified to enhance the interaction between MPs and host cells, to produce biopolymers that can possibly enable MP upcycling as biopolymer-MP composite, or to encode plastic degrading enzymes for microplastic degradation. Heinemann et al., 2006. “Synthetic biology—putting engineering into biology” Bioinformatics 22:2790-2799; Roh et al., 2021. “Improved CO2-derived polyhydroxybutyrate (PHB) production by engineering fast-growing cyanobacterium Synechococcus elongatus UTEX 2973 for potential utilization of flue gas” Bioresource Technology 327; Ciebiada et al., 2020. “Modifying the Cyanobacterial Metabolism as a Key to Efficient Biopolymer Production in Photosynthetic Microorganisms” International Journal of Molecular Sciences 21; Wang et al. 2020. “Enhancing secretion of polyethylene terephthalate hydrolase PETase in Bacillus subtilis WB600 mediated by the SPamy signal peptide” Lett Appl Microbiol 71:235-241; Seo et al., 2019. “Production of extracellular PETase from Ideonella sakaiensis using sec-dependent signal peptides in E-coli” Biochemical and Biophysical Research Communications 508:250-255; and Huang et al., 2018. “Tat-Independent Secretion of Polyethylene Terephthalate Hydrolase PETase in Bacillus subtilis 168 Mediated by Its Native Signal Peptide” Journal of Agricultural and Food Chemistry 66:13217-13227.

In such a way, MPs in the wastewater could be effectively removed and, at the same time, be either upcycled or degraded, and thus will less likely re-enter into the ecosystem. On the other hand, bioremediation by some photosynthetic microbes can remove excessive nutrients from wastewater simultaneously, providing extra benefits to the treatment process. Ghimire et al., 2017. :Bio-hythane production from microalgae biomass: Key challenges and potential opportunities for algal bio-refineries” Bioresour Technol 241:525-536; and Ahmad I Z., 2022. “The usage of Cyanobacteria in wastewater treatment: prospects and limitations” Lett Appl Microbiol 75:718-730.

The data presented herein defines an innovative MP bioremediation strategy that can remove MPs and excessive nutrients simultaneously. In one embodiment, the bioremediation system comprises manipulating cell hydrophobicity of a fast-growing cyanobacterium to enhance cell-MP interaction. Although it is not necessary to understand the mechanism of an invention, it is believed that the bioremediation system results in a highly efficient MP removal from both potable and wastewater. In one embodiment, the present invention contemplates an MP upcycling method comprising combining the MPs extracted from sediments and/or cyanobacterial endogenous biopolymers to produce bioplastic composites.

### A. MP Capture By Increasing Cyanobacterial Cell Hydrophobicity

Hydrophobic interaction is one of the major driving forces for biotic and abiotic interactions. Given the hydrophobic property of most plastics, it is possible to take advantage of such interaction for NP and MP remediation. As discussed above, increased cell hydrophobicity of cyanobacteria has been achieved by engineering cyanobacteria to overexpress hydrophobic compounds such as limonene (e.g., L524). Engineered strain L524 cells aggregate as a result of the hydrophobic effect when agitation is stopped. Such aggregation leads to highly efficient sedimentation and was proposed to be a cost-effective method for cyanobacterial biomass collection. Long et al., 2022. “Machine learning-informed and synthetic biology-enabled semi-continuous algal cultivation to unleash renewable fuel productivity” Nature Communications 13.

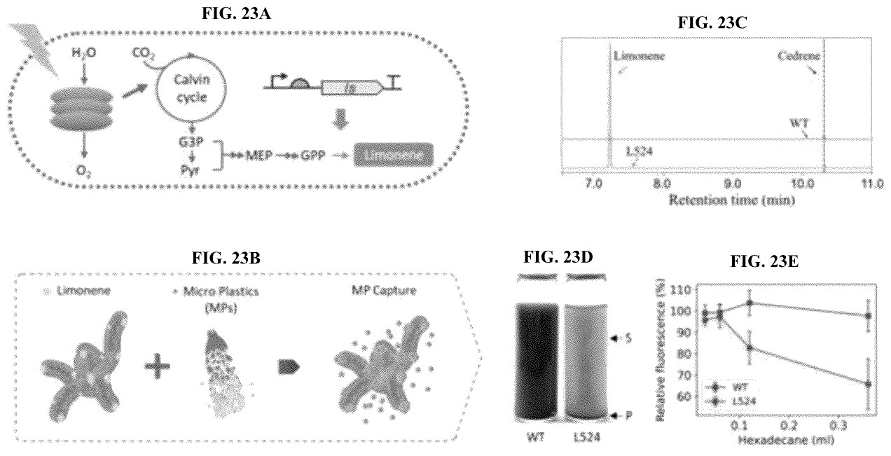

Although it is not necessary to understand the mechanism of an invention, it is believed that engineered high-hydrophobicity cyanobacterial cells could capture NPs and MPs and form plastics-cyanobacteria aggregates, which can be settled subsequently. FIG. 23B. To test the hypothesis, an overexpressing Mentha spicata limonene synthase was overexpressed in Synechococcus elongatus UTEX 2973 (UTEX 2973 thereafter). FIG. 23A. The limonene production of UTEX 2973 was verified by gas chromatography-mass spectrometry. FIG. 23C. The increased cell hydrophobicity of UTEX 2973 was verified by bacterial adherence to hydrocarbons (BATH) assay. FIG. 23E.

Specifically, substantially more limonene-producing cells were observed to be attached to the hydrocarbon layer, resulting lower chlorophyll florescence in the water phase as compared to wildtype (WT). FIG. 23E. As a result, efficient auto-sedimentation was found in the limonene producing strain and is believed to be useful for NP and MP bioremediation. FIG. 23D.

### B. MP Bioremediation By UTEX 2973

Given the increased cell hydrophobicity property due to limonene production, it was believed that the engineered L524 cyanobacteria could effectively capture MPs (e.g., polystyrene nanoparticles; PS NPs). Polystyrene nanoparticles ranging from 200 nm to 5 pm were evenly distributed a suspension, which ensures measurement consistency. The density of PS nanoparticles (around 1.05 g/ml) is very close to water, which allows them to stay in suspension for extended time and have slow sedimentation rates. As a result, removal efficiency can be determined by measuring PS NPs remaining in suspension. Lastly, PS NP suspensions are naturally in white color, which allows simplified quantification by measuring suspension turbidity.

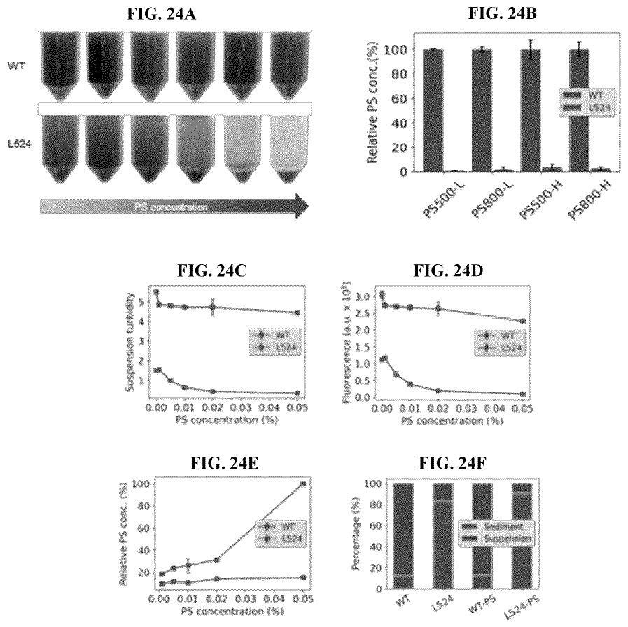

A gradient concentration of 200 nm PS nanoparticles were mixed with WT or L524 cells. Significant limonene-mediated cell aggregation/sedimentation was observed in L524 but not in WT samples after settling for 1 h. Sedimentation was accelerated and enhanced by PS microplastics, implying the presence of potential cell-PS interaction. FIG. 24A.

Detailed analysis on the suspensions revealed substantially lower turbidity, chlorophyll fluorescence, and PS concentration in L524 samples than in WT samples, highlighting the effectiveness of PS removal by the limonene producing strain. FIGS. 24C, D & E. Furthermore, similar capacity was also observed in the removal of 500 nm and 800 nm PS particles, suggesting that the limonene producing cells can capture PS MPs with a wide range of sizes. FIG. 24B.

PS NP removal efficiency was quantified as dry weight of suspensions and sediments in both WT and L524 samples. Due to the absence of sediments in WT samples, the bottom 5 ml of the samples were collected for both WT and L524 cells. Such a sampling strategy is likely to lead to overestimation of WT (and WT-PS) sedimentation but its impacts on L524 samples should be limited as a result of the relative low cell concentrations in L524 suspensions. FIG. 23A. The data show that 11.9% and 82.5% of WT and L524 cells, respectively, are sedimented after 1 h settling when PS MPs are absent. The numbers increase to 12.7% and 90.3% when cells were mixed with PS particles. FIG. 24F.

These results suggested an efficient sedimentation and potential for MP bioremediation removal with L524 cells but not in WT cells. It is believed that the observed MP removal capacity greater than any conventional biological plastic bioremediation system. The high MP removal capacity allows the presently disclosed system to be applied to scenarios containing a wide range of MP concentrations. Taken together, the results revealed a robust MP bioremediation capacity of cyanobacteria that overexpress limonene.

### C. Hydrophobic Interactions Between Microplastics And Cyanobacteria

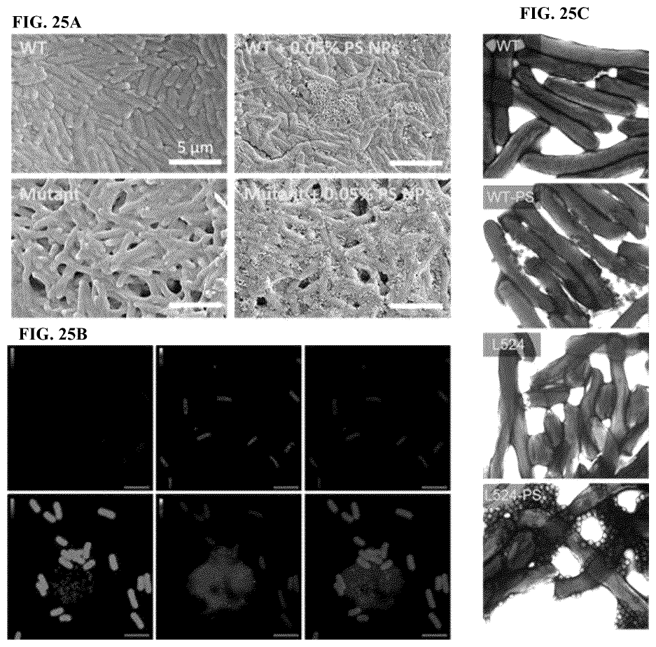

Limonene-mediated the PS nanoparticle removal by L524 was further analyzed by determining the interaction(s) between PS nanoparticles and cyanobacterial cells with a scanning electron microscope (SEM). The data show that PS nanoparticles were found to be uniformly attached to the L524 cell surface. By contrast, MPs showed a varied WT cells attachment distribution with significant PS aggregates in the suspension. FIG. 25A.

The uniform attachment of PS nanoparticles on the L524 cells indicates an active capacity of PS capturing. It is worth noting that the presence of PS nanoparticles on WT samples might not reflect a real MP capture capacity of WT cells. For example, freeze-drying process (e.g., lyophilization) in SEM sample preparation might push the PS particles (in)to WT cells.

Thus, transmission electron microscopy (TEM) was used to verify the SEM results which has the advantage of a fast negative stain procedure and less volume. Consequently the TEM observations are more representative of a real PS-cell interactions in solution. Indeed, the TEM data shows fewer PS particles attached to the WT cells as compared to the L534 cells and such attachment is randomly distributed. Conversely, significantly larger number of PS particles were found attached to L524 cells. In particular, significant PS enrichment was found at L524 cell intersections, where limonene is expected to be present to mediate cell aggregations. FIG. 25C. These observations support a hypothesis that limonene is the potential driving force of the MP removal.

To further validate the involvement of limonene in the PS nanoparticle removal, a stimulated Raman scattering microscopy (SRS) was used to visualize an interaction between limonene and PS nanoparticles. Strong intracellular and extracellular limonene signals (green, first column) were observed in L524 samples, while such a signal was not detected in WT. FIG. 25B. Meanwhile, PS nanoparticles were visualized at 2900 cm⁻¹ CH₂ frequency.

Although a weak signal was found in cyanobacterial cells, it was seen as background due to the presence of CH₂ bonds in cyanobacteria. FIG. 25B; second column. In contrast, strong CH₂ signal was observed in PS particles. FIG. 25B; red—second column. Overlaying the signals revealed direct interactions between limonene and PS particles. FIG. 25B; third column, These data indicate limonene as a mediator induced the PS aggregation and capturing. Overall, these results validated the enhanced MP capture capacity of L524 cells and identified limonene as a mediator for the MP capture.

Hydrophobic interactions between cyanobacteria cells and PS particles were evaluated with surfactant-mediated blocking experiments. Specifically, Tween 20 was introduced to block hydrophobic surfaces on both cyanobacterial cells and PS particles. With the treatment, an increased suspension turbidity was observed in the L524 sample, indicating a suppressed cell aggregation and disabled aggregation-based sedimentation. (data not shown). Interestingly, the addition of Tween 20 also slightly increased WT suspension turbidity, suggesting that Tween 20 slowed down the settling of WT cells.

It is worth mentioning that the samples for turbidity measurements were taken from the top of suspension. No visible sedimentations were observed in the bottoms of WT samples despite the lower turbidity as compared to the WT-Tween 20 samples. Similar results were found in samples in the presence of PS particles, indicating inhibitory effects of the surfactant on MP-cell interaction. More importantly, the contents of PS particles were found to be significantly higher when Tween 20 was added, in both WT and L524. (data not shown). The results highlight that hydrophobic interactions play a role in MP-mediated bioremediation using cyanobacteria that overexpress limonene.

### D. Spectrum: L524 Cell-Microplastics Interactions

Cyanobacteria overexpression limonene was evaluated for interactions with other types of MPs, namely polyethylene terephthalate (PET) and polyethylene (PE). The dispersibility of PET and PE in water is low, presumably due to their large size (<300 micron for PET and 32-38 micron for PE). Thus, a sedimentation assay used for PS removal quantification would not be expected to provide reliable data to determine PET-cell and PE-cell interactions.

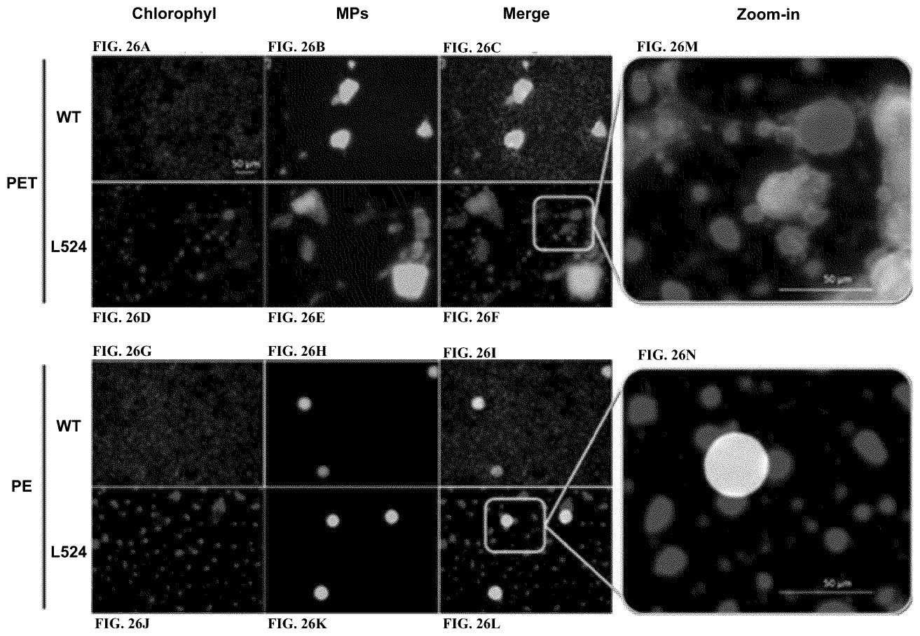

One method to evaluate limonene overexpressing cyanobacteria capture of PET and PE is direct microscopy observation. Cyanobacterial cells were visualized with chlorophyll fluorescence (red) and PET and PE autofluoresence (cyan). Cell aggregates were observed in L524 samples but not in WT samples, which is consistent with the previous study (33). FIGS. 26D & J cf FIGS. 26A & G.

When an overlay of chlorophyll fluorescence of cyanobacteria with the auto-fluorescence of PET and PE is evaluated, cell-MP interactions were observed in PET-L524 and PE-L524 samples but few interactions were observed in PET-WT and PE-WT samples. FIGS. 26F & L cf FIGS. 26C & I. In particular, zoom-in images clearly showed the attachment of cyanobacterial cells/aggregates on PET and PE microplastics. FIGS. 26M & N.

Together, these results validated that interactions between limonene producing cells and MPs suggest that the engineered cyanobacteria useful for bioremediation for a wide spectrum of MPs.

### E Upcycle Microplastics as Bioplastic Composites

One of the biggest challenges in bioremediation is the long persistence of MPs. MPs removed from remediation processes might be able to reenter the ecosystems if there is lack of an appropriate downstream treatment method. One advantage of a cyanobacterium-based bioremediation system is its ability of integrating into various downstream biological modules for MP upcycling or degradation.

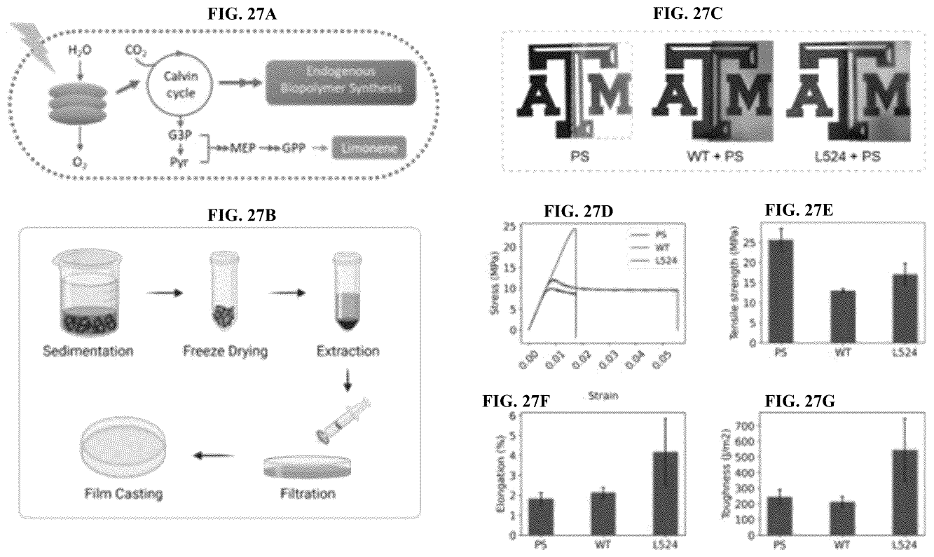

In one embodiment, the present invention contemplates an MP upcycling method comprising combining endogenous cyanobacterial biopolymers and MPs to produce a bioplastic composite. FIG. 27A. In one embodiment, the bioplastic composite is a replacement for petroleum-based plastics. In one method, PS-L524 mixtures were sedimented and transformed into bioplastic films via casting. FIG. 27B. These methods have successfully created a bioplastic film from both WT-PS and L524-PS sediments. FIG. 27C.

In one embodiment, the present invention contemplates a polystyrene (PS) bioplastic film created from a pure polystyrene foam and cyanobacterial polymers. In one embodiment, the PS bioplastic film comprised a golden-green color. FIG. 27C. A spectrum analysis revealed the presence of absorbance peaks similar to chlorophyll A and carotenoid in the bioplastics suggesting that these pigments were extracted and casted into the bioplastic during the film preparation. FIG. 25C. It was further observed that higher absorbance peaks suggest higher content of cyanobacterial biomass in L524-PS sediments than WT-PS, due to the limonene-induced sedimentation (data not shown).

Mechanical performance of bioplastic composites were determined, using a pure polystyrene film as a control. Composite bioplastic films made from WT-PS and L524-PS sediments showed distinct mechanical properties as compared to pure PS films. FIG. 27D. Both bioplastic composite films showed a lower tensile strength, only 52.9% and 66.5% as compared to pure PS films for WT-PS and L524-PS bioplastics, respectively.

While no significant (p<0.05) differences were observed between pure PS and WT-PS films in terms of modulus of elasticity, elongation, and toughness, these properties were found to be significant improved in the L524-PS films. In particular, the elongation and toughness of the L524-PS bioplastics were 2.3 and 2.2-folds higher compared to the PS film, indicating superior properties of the upcycled bioplastics. Such improvement could result from the addition of biopolymers, which altered the chemical composition and/or linkages of the polymer mixture. On the other hand, the small amount of sulfate residue (for the MP preparation) on the MP surface might also contribute to the mechanical property changes. Overall, these results demonstrate that MP upcycling may be integrated with engineered microorganisms to create improved bioremediated plastic composite compounds.

### F. Combination Plastics And Excessive Nutrient Removal

In one embodiment, the present invention contemplates a method comprising removing both microplastics and excessive nutrients from a wastewater with a cyanobacteria over-expressing a limonene synthase.

Cyanobacteria has been proposed to be a promising strategy for wastewater treatment to deplete excessive nutrients. Sood et al., 2015. “Cyanobacteria as Potential Options for Wastewater Treatment”, p 83-93. In: Ansari A A, Gill S S, Gill R, Lanza G R, Newman L (ed), Phytoremediation: Management of Environmental Contaminants, Volume 2; Springer International Publishing, Cham.

A limonene-producing strain is tested to show remediation of excessive nutrients and MPs from wastewater simultaneously.

### G. Commercial Advantages

The presently disclosed invention is fundamentally different from conventional bioremediation processes. Instead of relying on extracellular polymeric substance (EPS) production and biofilm formation, MP capture capacities of cyanobacteria are directly enhanced by increasing cell hydrophobicity by engineering cells to overexpress limonene production. See, Table 4.

While the MP removal efficiencies are unavailable for the majority of these conventional processes, the presently disclosed system is based upon results from a comprehensive analyses, including, but not limited to, turbidity measurements, imaging visualizations, SRS, and TAG a;; which indicate that the limonene-based plastic capture is fast and highly effective. In particular, the cyanobacteria UTEX 2973 is expected to produce low amount of EPS, thereby explaining the negligible sedimentation observed in UTEX 2973 WT samples. de Oliveira et al., 2020. “Interaction of Cyanobacteria with Nanometer and Micron Sized Polystyrene Particles in Marine and Fresh Water” Langmuir 36:3963-3969; and Yu et al., 2015. “Synechococcus elongatus UTEX 2973, a fast growing cyanobacterial chassis for biosynthesis using light and CO₂. Scientific Reports 5.

Thus, the highly effective MP capture by the limonene producing strains represents a universal strategy for cell hydrophobicity manipulation and MP remediation engineering using microorganisms. Further, one engineered strain captured a variety of MPs, including PE, PET, and PS. Given the hydrophobic nature of majority plastics, it is reasonable to expect that most, if not all MPs can undergo bioremediation using this strategy. This is particularly advantageous because MPs with lower densities and buoyant liquid properties, such as PS and PE, are difficult to remove by sedimentation. Indeed, no noticeable sedimentations were observed in PE particles and PS particles with diameters less than 1 pm when cyanobacterial cells were absent (data not shown). The data presented herein regarding hydrophobicity-mediated aggregation is an effective solution to non-sedimentation remediation processes, as substantial portions of PE and small-size PS MPs were removed by the limonene producing cyanobacteria.

Together with a highly efficient MP removal, the present invention contemplates an MP upcycling strategy to transform the removed MPs together with cyanobacterial biomass into bioplastic composites. In one embodiment, the present invention contemplates a method comprising reducing global MP generation by replacing non-degradable petro-based plastics with degradable bio-based composite polymers.

Bioplastics produced from cyanobacteria-MP sediments comprise both recycled MPs and cyanobacterial biodegradable polymers which may also contain proteins, lipids, polysaccharides, etc. Despite the decreased tensile strength of the bioplastic composites made from the L524-PS aggregates, their modulus of elasticity, elongation, as well as toughness were all found to be enhanced (supra), implying that replacing petro-based plastics would be advantageous.

Further optimization of a bioplastic composite preparation process, such as using alternative extraction solvents and/or casting protocols, would be expected improve uniformity and mechanical performance of the bioplastic compositions. For example, UTEX 2973 has been successfully engineered to produce polyhydroxybutyrate (PHB), which is a widely used biopolymer for bioplastics. Roh et al., 2021. “Improved CO2-derived polyhydroxybutyrate (PHB) production by engineering fast-growing cyanobacterium Synechococcus elongatus UTEX 2973 for potential utilization of flue gas” Bioresource Technology 327.

Similar bioengineering strategies can be applied together with the limonene biosynthesis to enable the effective MP removal and potential enhance the mechanical performance of the upcycled bioplastic composite. On the other hand, plastic depolymerization pathways can be also installed to the UTEX 2973 to accelerate MP degradation at scenarios where upcycling is impossible. Tournier et al., 2020. “An engineered PET depolymerase to break down and recycle plastic bottles' Nature 580:216-+; Yoshida et al.,. 2016. ‘A bacterium that degrades and assimilates poly(ethylene terephthalate)” Science 351:1196-1199; Lu et al.,. 2022. “Machine learning-aided engineering of hydrolases for PET depolymerization” Nature 604:662-+; and Chen et al.,. 2022. “Biodegradation of highly crystallized poly(ethylene terephthalate) through cell surface codisplay of bacterial PETase and hydrophobin” Nature Communications 13.

Overall, the data presented herein demonstrating hydrophobicity-based bioremediation represents not only a highly efficient method to remove a wide spectrum of MPs, but also a flexible platform that compatible with a variety of downstream MP upcycling and treatment strategies.

### SUPERSCRIPTED REFERENCES

1. Wang X, Ort D R, Yuan J S. Photosynthetic terpene hydrocarbon production for fuels and chemicals. Plant Biotechnol J 13, 137-146 (2015).
2. Singh N K, Dhar D W. Microalgae as second generation biofuel. A review. Agron Sustain Dev 31, 605-629 (2011).
3. Milledge J, Heaven S. A review of the harvesting of micro-algae for biofuel production. Rev Environ Sci Bio 12, 165-178 (2013).
4. Rawat I, Kumar R R, Mutanda T, Bux F. Biodiesel from microalgae: A critical evaluation from laboratory to large scale production. Appl Energ 103, 444-467 (2013).
5. Barros A I, Goncalves A L, Simoes M, Pires J C M. Harvesting techniques applied to microalgae: A review. Renew Sust Energ Rev 41, 1489-1500 (2015).
6. Gupta P L, Lee S M, Choi H J. A mini review: photobioreactors for large scale algal cultivation. World J Microb Biot 31, 1409-1417 (2015).
7. Lam M K, Lee K T. Microalgae biofuels: A critical review of issues, problems and the way forward. Biotechnol Adv 30, 673-690 (2012).
8. Mata T M, Martins A A, Caetano N S. Microalgae for biodiesel production and other applications: A review. Renew Sust Energ Rev 14, 217-232 (2010).
9. Wang J F, Liu J L, Liu T Z. The difference in effective light penetration may explain the superiority in photosynthetic efficiency of attached cultivation over the conventional open pond for microalgae. Biotechnol Biofuels 8, (2015).
10. Lee C-G. Calculation of light penetration depth in photobioreactors. Biotechnology and Bioprocess Engineering 4, 78-81 (1999).
11. Comet J F, Dussap C G, Gros J B, Binois C, Lasseur C. A Simplified Monodimensional Approach for Modeling Coupling between Radiant Light Transfer and Growth-Kinetics in Photobioreactors. Chem Eng Sci 50, 1489-1500 (1995).
12. Katsuda T, et al. Light intensity distribution in the externally illuminated cylindrical photo-bioreactor and its application to hydrogen production by Rhodobacter capsulatus. Biochem Eng J 5, 157-164 (2000).
13. Kumar K, Sirasale A, Das D. Use of image analysis tool for the development of light distribution pattern inside the photobioreactor for the algal cultivation. Bioresource Technol 143, 88-95 (2013).
14. Suh I S, Lee S B. A light distribution model for an internally radiating photobioreactor. Biotechnol Bioeng 82, 180-189 (2003).
15. Dassey A J, Theegala C S. Harvesting economics and strategies using centrifugation for cost effective separation of microalgae cells for biodiesel applications. Bioresource Technol 128, 241-245 (2013).
16. Singh G, Patidar S K. Microalgae harvesting techniques: A review. J Environ Manage 217, 499-508 (2018).
17. Vapnik V, Golowich S E, Smola A. Support vector method for function approximation, regression estimation, and signal processing. Adv Neur In 9, 281-287 (1997).
18. Kwok J T Y. Support vector mixture for classification and regression problems. Int C Patt Recog, 255-258 (1998).
19. Smola A J, Scholkopf B. A tutorial on support vector regression. Stat Comput 14, 199-222 (2004).
20. Yen H W, Chiang W C. Effects of mutual shading, pressurization and oxygen partial pressure on the autotrophical cultivation of Scenedesmus obliquus. J Taiwan Inst Chem E 43, 820-824 (2012).
21. Clark R L, et al. Light-optimized growth of cyanobacterial cultures: Growth phases and productivity of biomass and secreted molecules in light-limited batch growth. Metabolic Engineering 47, 230-242 (2018).
22. Simionato D, Basso S, Giacometti G M, Morosinotto T. Optimization of light use efficiency for biofuel production in algae. Biophys Chem 182, 71-78 (2013).
23. Ooms M D, Dinh C T, Sargent E H, Sinton D. Photon management for augmented photosynthesis. Nat Commun 7, (2016).
24. Nwoba E G, Parleyhet D A, Laird D W, Alameh K, Moheimani N R. Light management technologies for increasing algal photobioreactor efficiency. Algal Res 39, (2019).
25. Yu J J, et al. Synechococcus elongatus UTEX 2973, a fast growing cyanobacterial chassis for biosynthesis using light and CO₂. Sci Rep-Uk 5, (2015).
26. Ungerer J, Lin P C, Chen H Y, Pakrasi H B. Adjustments to Photosystem Stoichiometry and Electron Transfer Proteins Are Key to the Remarkably Fast Growth of the Cyanobacterium Synechococcus elongatus UTEX 2973. Mbio 9, (2018).
27. Ungerer J, Wendt K E, Hendry J I, Maranas C D, Pakrasi H B. Comparative genomics reveals the molecular determinants of rapid growth of the cyanobacterium Synechococcus elongatus UTEX 2973. P Natl Acad Sci USA 115, E11761-E11770 (2018).
28. Liu Y, Yang S F, Tay J H, Liu Q S, Qin L, Li Y. Cell hydrophobicity is a triggering force of biogranulation. Enzyme Microb Tech 34, 371-379 (2004).
29. Wang X, et al. Enhanced limonene production in cyanobacteria reveals photosynthesis limitations. Proc Natl Acad Sci USA 113, 14225-14230 (2016).
30. Davies F K, Work V H, Beliaev A S, Posewitz M C. Engineering Limonene and Bisabolene Production in Wild Type and a Glycogen-Deficient Mutant of Synechococcus sp. PCC 7002. Front Bioeng Biotechnol 2, 21 (2014).
31. Lin PC, Saha R, Zhang F, Pakrasi HB. Metabolic engineering of the pentose phosphate pathway for enhanced limonene production in the cyanobacterium Synechocysti s sp. PCC 6803. Sci Rep 7, 17503 (2017).
32. Freudiger C W, et al. Label-free biomedical imaging with high sensitivity by stimulated Raman scattering microscopy. Science 322, 1857-1861 (2008).
33. Zhao C, et al. Co-Compartmentation of Terpene Biosynthesis and Storage via Synthetic Droplet. Acs Synthetic Biology 7, 774-781 (2018).
34. Rosenberg M. Bacterial Adherence to Hydrocarbons—a Useful Technique for Studying Cell-Surface Hydrophobicity. Fems Microbiol Lett 22, 289-295 (1984).
35. Packer M. Algal capture of carbon dioxide; biomass generation as a tool for greenhouse gas mitigation with reference to New Zealand energy strategy and policy. Energ Policy 37, 3428-3437 (2009).
36. Li SB, Sun T, Xu CX, Chen L, Zhang W W. Development and optimization of genetic toolboxes for a fast-growing cyanobacterium Synechococcus elongatus UTEX 2973. Metabolic Engineering 48, 163-174 (2018).
37. Lima NGPB, et al. Anxiolytic-like activity and GC-MS analysis of (R)-(+)-limonene fragrance, a natural compound found in foods and plants. Pharmacol Biochem Be 103, 450-454 (2013).
38. Hirota R, Roger N N, Nakamura H, Song H S, Sawamura M, Suganuma N. Anti-inflammatory Effects of Limonene from Yuzu (Citrus junos Tanaka) Essential Oil on Eosinophils. J Food Sci 75, H87-H92 (2010).
39. Hirota R, et al. Limonene inhalation reduces allergic airway inflammation in Dermatophagoides farinae-treated mice. Inhal Toxicol 24, 373-381 (2012).
40. Chuck C J, Donnelly J. The compatibility of potential bioderived fuels with Jet A-1 aviation kerosene. Appl Energ 118, 83-91 (2014).
41. Tracy N I, Chen D C, Crunkleton D W, Price G L. Hydrogenated monoterpenes as diesel fuel additives. Fuel 88, 2238-2240 (2009).
42. Song K, Tan X, Liang Y, Lu X. The potential of Synechococcus elongatus UTEX 2973 for sugar feedstock production. Appl Microbiol Biotechnol 100, 7865-7875 (2016).
43. Wlodarczyk A, Selao T T, Norling B, Nixon P J. Newly discovered Synechococcus sp. PCC 11901 is a robust cyanobacterial strain for high biomass production. Commun Biol 3, (2020).
44. Ryan Davis J M, Christopher Kinchin, Nicholas Grundl, Eric C. D. Tan. Process Design and Economics for the Production of Algal Biomass: Algal Biomass Production in Open Pond Systems and Processing Through Dewatering for Downstream Conversion. In: National Renewable Energy Laboratory) (2016).
45. Clippinger JNRED. Techno-Economic Analysis for the Production of Algal Biomass via Closed Photobioreactors: Future Cost Potential Evaluated Across a Range of Cultivation System Designs. (ed Energy USDo) (2019).
46. Clippinger J, Davis R. Techno-Economic Analysis for the Production of Algal Biomass via Closed Photobioreactors: Future Cost Potential Evaluated Across a Range of Cultivation System Designs. National Renewable Energy Lab(NREL), Golden, CO (United States), (2019).
47. Aikawa S, et al. Direct conversion of Spirulina to ethanol without pretreatment or enzymatic hydrolysis processes. Energ Environ Sci 6, 1844-1849 (2013).
48. Aikawa S, et al. Direct and highly productive conversion of cyanobacteria Arthrospira platensis to ethanol with CaCl₂) addition. Biotechnol Biofuels 11, 50 (2018).
49. Chojnacka K, Wieczorek P P, Schroeder G, Michalak I. Algae Biomass: Characteristics and Applications Towards Algae-based Products Preface. Devel Appl Phycol 8, V-Vi (2018).
50. Becker E W. Micro-algae as a source of protein. Biotechnology Advances 25, 207-210 (2007).
51. Zeng Y, Himmel M E, Ding S Y. Visualizing chemical functionality in plant cell walls. Biotechnol Biofuels 10, 263 (2017).
52. Badulescu R, Vivod V, Jausovec D, Voncina B. Treatment of Cotton Fabrics with Ethyl Cellulose Microcapsules. 226-235 (2010).
53. Claudino M, Jonsson M, Johansson M. Utilizing thiol-ene coupling kinetics in the design of renewable thermoset resins based on D-limonene and polyfunctional thiols. Rsc Adv 4, 10317-10329 (2014).
54. Halfmann C, Gu L P, Zhou R B. Engineering cyanobacteria for the production of a cyclic hydrocarbon fuel from CO2 and H2O. Green Chem 16, 3175-3185 (2014).
55. Jaiswal D, et al. Genome Features and Biochemical Characteristics of a Robust, Fast Growing and Naturally Transformable Cyanobacterium Synechococcus elongatus PCC 11801 Isolated from India. Sci Rep-Uk 8, (2018).
56. Jaiswal D, Sengupta A, Sengupta S, Madhu S, Pakrasi H B, Wangikar P P. A Novel Cyanobacterium Synechococcus elongatus PCC 11802 has Distinct Genomic and Metabolomic Characteristics Compared to its Neighbor PCC 11801. Sci Rep-Uk 10, (2020).
57. Pathania R, Srivastava S. Synechococcus elongatus BDU 130192, an Attractive Cyanobacterium for Feedstock Applications: Response to Culture Conditions. Bioenerg Res, (2020).

### Experimental

The following examples serve to illustrate certain embodiments and aspects of the present invention and are not to be construed as limiting the scope thereof.

In the experimental disclosures which follow, the following abbreviations apply: N (normal); M (molar); mM (millimolar); μM (micromolar); mol (moles); mmol (millimoles); p.mol (micromoles); nmol (nanomoles); pmol (picomoles); g (grams); mg (milligrams); gg (micrograms); ng (nanograms); pg (picograms); L and (liters); ml (milliliters); μl (microliters); cm (centimeters); mm (millimeters); gm (micrometers); nm (nanometers); U (units); min (minute); s and see (second); deg (degree); ° C. (degrees Centigrade/Celsius).

### Example I

### Primary Methods

### 1. Strains and Growth Conditions

S. elongatus UTEX 2973 wild-type, endogenously expressing Geranyl pyrophosphate synthase (GPPS), responsible for the formation of geranyl pyrophosphate (GPP), was obtained from Dr. Pakrasi at Washington University. Strains were maintained in BG11 plates and liquid (Sigma, C3061) supplemented with 10 mM TES under 50 μmol photons m⁻² s⁻¹ illumination at 37° C. A customized PBR (based on a 1-L Roux bottle) containing 500 ml of media was used for cultivation, with 5% (vol/vol) CO₂ bubbling from a stainless-steel aeration stone at a speed of 0.8 L/min. 10 ml of 50× stock media was fed every 24 hours for fed-batch (FB) cultivation. For SAC, initial cell concentration was adjusted to OD₇₃₀ of ˜2.3 every 24 hours followed by media feeding. The initial OD was selected based on the machine learning model outcome of optimal starting OD. The growth temperature of batch cultivation, fed-batch, and SAC was maintained at 37° C. Artificial light at 574 μmol m⁻² s⁻¹ was applied on two opposite sides of the PBR, after initial growth with one-side 357 μmol m⁻² s⁻¹ and 714 μmol m⁻² s⁻¹ at 0-12 h and 12-36 h, respectively.

2. Bioplastic film production from NPs and L524

Procedure: 1) 0.1 g/L 800 nm polystyrene beads was mixed with 35 ml WT and L524 (OD=15). Let the mixtures settle for 1 h, and collect the bottom 5 ml for centrifugation (7000 rpm for 10 min). The pellets are lyophilized before adding 3 ml chloroform to each tube. Polystyrene beads and cells were dissolved with sonication (10 s on/50 s off for 6 cycles) and the solvent were centrifuged with 10000 rpm for 10 mins. The supernatants were filtrated with 0.2 um PTFE filter (need several filters for complete filtration) before casting film.

Engineered Synechococcus elongatus UTEX 2973, i.e. strain L524, itself aggregates by auto-sedimentation. Therefore, aggregation occurs no matter how low the concentrated NPs added to solution. However, higher NP concentration accelerates sedimentation, See e.g., FIGS. 1-2, and may be used to successfully make films. On the other hand, due to the stronger capacity of L524 cells in capturing PS NPs, The film made from L524+PS mixture has better mechanical properties than WT+PS mixture. In the demonstration experiments, 0.3% 800 nm PS particles were used. 0.3% 200 nm, 500 nm, PS NP were also tested. It does not matter whether cyanobacteria are added before or after NPs.

### 3. Bioplastic Film Production Using Cyanobacteria as Composite Enforcer

Bioplastics were made from sediments containing cyanobacteria and PS particles. Specifically, 35 ml of cyanobacteria were mixed with 1 ml of 10% PS stock (800 nm) and then let the mixture settle for 1 h. The bottom 5 ml of samples was collected as sediments and lyophilized for 24-48 hours until they dry completely. The mixtures were then dissolved in chloroform with sonication (10 s on/50 s off for 6 cycles), followed by centrifugation at 5700×g for 10 mins. The supernatants were filtrated with a 0.2 um PTFE filter and casted in aluminum plates.

Procedure: 1) sonicate cyanobacterial cells to break down cell compartments; 2) mix cyanobacterial lysate with CMC solution at a ratio of 1:1 (mass: mass); 3) casting the mixture at 40 Celsius. See, FIG. 5.

Applications: There are numerous potential applications of such films (including a composite enforcer, e.g., CMC), especially after some future modifications. One example demonstrated that CMC type bioplastic may be used as mulch, given the properties of biodegradability of CMC and light blackout.

### 4. Microscopy Imaging and Aggregation Evaluation

Cells sampled from cyanobacterial culture were adjusted to the same concentrations and transferred to Eppendorf tubes for aggregation. After 30 minutes, the tubes were gently vortexed to suspend pellets (in L524) while minimizing the perturbation for aggregation. The well-mixed samples were observed under Leica DM6B. For cell aggregation quantification, the well-mixed samples were counted with a hemocytometer. Cell aggregation was defined as aggregates with five or more cells. The number of aggregated L524 cells was estimated by subtracting the number of unaggregated L524 cells from WT cells. In the transmission electron microscopy (TEM) observation, cells were negatively stained with 1% uranyl acetate and observed under JEOL 1200. Scanning electron micrographs (SEM) show bacteria prepared using standard methods.

### 5. Chemical Imaging Analysis

SRS microscopy developed for plant biomass imaging was used to perform the chemical imaging.⁵¹ A HighQ picoTRAIN (Spectra-Physics) laser was used to generate 1064 nm (up to 15 W) and 532 nm (up to 9 W) output; both are pulse trains at 7 ps. The 1064 nm output was used as the SRS Stokes beam. The 532 nm beam was used to pump an APE optic parametric oscillator (Levante Emerald, APE GmbH, Germany) to produce a tunable wavelength 6 ps pulse train to be used as the SRS pump beam. The 1064 nm Stokes beam was modulated by an acoustic optical modulator (3080-122, Crystal Technology) at 10 MHz frequency, achieving >80% intensity modulation depth. Both the pump and Stokes pulse trains were combined (1064dcrb, Chroma) and routed to a modified scanner (BX62WI/FV300, Olympus) attached to an Olympus IX81 microscope. The pump beam intensity after the sample was collected by a high numeric aperture lens, filtered and detected by a photodiode. A lock-in amplifier was used to detect the stimulated Raman loss signal. The Raman frequency of the limonene C═C bond at 1670 cm⁻¹ that was previously used by other studies^{33, 52, 53} was chosen for SRS imaging, which corresponded to a pump wavelength at 903 nm.

### 6. Aggregation-Based Sedimentation Measurement

The efficiency of ABS was assessed by monitoring the sedimentation process of cyanobacterial cells (OD₇₃₀ at 10.0) in a harvesting vessel with a 20-centimeter height. Cell concentrations on the surface were used to evaluate the sedimentation efficiency. The vertical distribution of cyanobacteria was evaluated by sampling cells at different depths with a long glass tip.

### 7. Bacterial Adherence to Hydrocarbon Assay

The bacterial adherence to hydrocarbon (BATH) assay was performed following the protocol developed by Rosenberg et al. with minor modifications.³⁴ Specifically, 3 ml of cyanobacteria with OD₇₃₀ of 0.2 were mixed with 0.12 ml of hexadecane. After phase separation, the chlorophyll fluorescence of the cyanobacteria (water phase) was measured to quantify cells that did not adhere to the hydrocarbon.

### 8. Limonene Collection and Measurement

Limonene produced from L524 was collected with HayeSep porous polymer (Sigma) head caps and eluted with 1 ml hexane containing 50 ppm cedrene (Sigma) as an internal standard (48). The elution was then analyzed by gas chromatography-mass spectrometry (GC-MS) (Shimadzu Scientific Instruments, Inc.). The GCMSsolution software (Version 4.11 SU2, Shimadzu Corporation) was used for peak integration, spectrum identification, and limonene quantification. A standard curve containing 125, 250, 500, 1000, 2000 ppm limonene standards was applied for limonene calculation.

### 9. Biomass Productivity Measurement

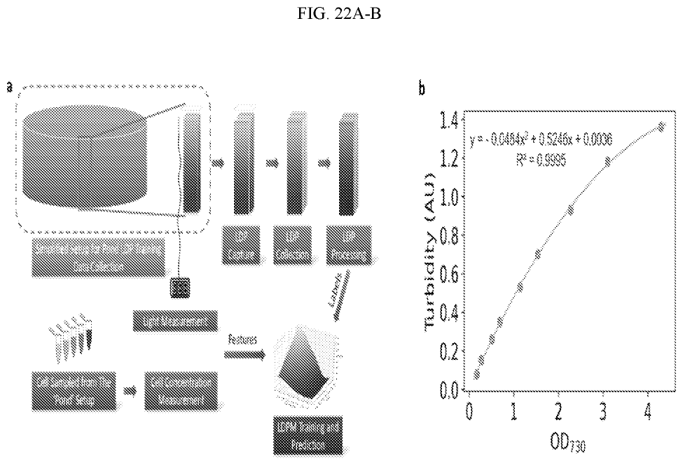

The biomass productivity was measured with OD₇₃₀ and converted to dry cell weight (DCW) with a pre-established calibration (1.0 OD₇₃₀ equals approximately 0.39 g DCW L⁻¹). The total biomass yields were calculated by adding the productivities of each day together. The biomass productivity from the pond system was calculated by first transforming the turbidity (Attenuation Unit, AU) to OD₇₃₀ with a calibration curve and then calculated as described above. See, FIG. 22B.

### 10. Limonene Production

Brief isolation method of limonene from cyanobacteria is by routine distillation techniques.

### 11. Techno-Economic Analysis

The techno-economic analysis was based on the algae farm model presented by NREL⁴⁴. Similar to the NREL study, we assumed the yearly biomass productivity to be the same as productivity achieved in Fall and set it to 43.3 g/m²/d. The 50-acre individual pond size was selected for the analysis and the pond harvest concentration was set to 0.7 g/L, as the SAC output (with initial cell concentration of 0.4 g/L) was about 0.7 g/L. The primary, secondary, and tertiary dewatering outlet concentrations were set to 140 g/L, according to the ABS output concentration. We set the ash content to 5.5% and used default values for the rest of the parameters in the analysis.

### 12. Pond Production

Calculate the amount of L524 inoculation, we need to use LDPM and GRM to simulate cyanobacterial growth under certain growth conditions. The simulation process involves three repeating steps:
i. Predicting LDP using LDPM (cell concentration, light intensity as inputs)
ii. Predicting growth rates using GRM (LDP as input)
iii. Updating cell concentration (cell concentration+growth rate*time) and light intensity (if there is a light program)
   After finishing step 3, repeat the cycle from step 1 until the simulation is completed. Inoculum amount changes depending upon growth conditions and purpose. For example, inoculum for a sunny day and cloudy day might be different for optimal biomass productivity. Once a growth pond is harvested, it may be reused without inoculation for reharvest. In one embodiment, a pond may be reinoculated as needed, depending upon environmental conditions and desired harvest times.

### 13. Pond Harvesting Methods

The amount of water removed from a pond or outdoor growth area by a water pump depends on one or more of: cell concentration, growth conditions, and also economics analysis. In the PBR setting, we harvest more than half of the water due to the high cell concentration and fast growth. However, less than half of the water is removed from pond systems.

### 14. Data Availability

Training data for machine learning models are available from GitHub (github.com/joshuayuanlabl51/LDPM-and-GRM).

### 15. Software Code Availability

Software code used for machine learning models and training data are available from GitHub website: github.com/joshuayuanlabl5l/LDPM-and-GRM. A stable release is available from: zenodo.org/badge/latestdoi/430008654.

### Example II

### Training Data Collection and Processing for Light Pattern Prediction

In order to access real-time light availability inside algal culture, we first developed a light distribution pattern prediction model (LDPM) to predict light distribution patterns (LDPs) in a cuboid photobioreactor (PBR). However, this method is not limited in cuboid PBRs, example extended to bodies of water. We collected 138 LDPs in the PBR (19.6 cm in length×9.6 cm in width×20 cm in height) with 23 different cell concentrations and 6 different light intensities as training samples for the machine-learning model. The LDPs were captured by a camera fixed on top of a PBR containing different cell concentrations and illuminated with different light intensities. A LED light bar (4000K, CRI=80) placed on one side of the photobioreactor was used as a light source (FIG. 6A). The illuminance was monitored by a sensor on the surface of the photobioreactor and converted to photosynthetic photon flux density (PPFD) with a coefficient of 56. The twenty-three cell concentration gradients were set to 0.11973, 0.21294, 0.40872, 0.45162, 0.54405, 0.62712, 0.74256, 0.82056, 0.90948, 0.96915, 1.10604, 1.2246, 1.3026, 1.3923, 1.443, 1.5444, 1.7901, 1.9188, 2.0241, 2.3556, 2.535, 2.9601, 3.6777 g/L, while the six light intensity gradients were set to 107, 178, 267, 357, 570, 714 μmol m⁻² s⁻¹. The camera was set to manual mode and all parameters were locked throughout the photographing process to ensure consistency. After acquiring all LDPs, raw pictures were cropped, converted to grayscale, and compressed to 40×18 pixels in Photoshop 2020 (FIG. 6A). The compressed images were used to represent the light distribution pattern inside the photobioreactor with grayscale values (GSVs) representing light intensities. The GSVs were extracted from the grayscale images with the CV2 module in Python. To evaluate the accuracy of GSVs representing light intensities, we extracted GSVs at (0, 20) (row 0, column 20) from LDPs over a wide range of cell concentrations and assessed the linearity between GSV and light intensity.

The training sample collection for pond LDPM. LDP images were captured from a simplified pond setup and the collected LDP images were converted to 208×10-pixel grayscale images as mentioned above. The light intensities for the pond LDP training samples were set to 196, 268, 357, 446, 536, 625, 714, 804, 964, 1071, 1161, 1250, 1339, 1429, 1518, 1607, 1696, 1786, 1875 μmol m⁻² s⁻¹, and cell concentrations were set to 0.062, 0.140, 0.228, 0.337, 0.466, 0.620, 0.871, 0.999, 1.177, 1.254, 1.396, 1.482, 1.553, 2.007, 2.320, 2.814, 3.199, 3.694, 4.577, 5.519 g/L. The LDP for the pond system was set to be one-dimensional and represented with the 208 pixels in the middle column (column 5) of the LDP image.

### Example III

### LDPM Training and Evaluation

Due to the high complexity of the LDP inside algal culture and limited training samples, we believe that predicting LDPs pixel by pixel is the best method for accurate prediction. Pixel-by-pixel prediction means that individual pixels in LDP images are treated as individual models and then combined, rather than treating the whole image as one model. Thus, we trained 720 models for a 40×18-pixel LDP prediction. Cell concentrations and light intensities, two major factors shaping LDP, were set as features in training with the corresponding GSVs at each pixel as labels. Both features and labels were normalized by subtracting their average and dividing by their standard deviation. Around 10% of the training samples were randomly selected as testing samples. We chose Support Vector Regression with a Radial Basis Function kernel (SVR-RBF) as the algorithm and kernel for the prediction in this study. SVR-RBF from an open-source machine learning library, scikit-learn¹, was used for training and prediction. We selected the best models at each pixel by selecting the combinations of parameters (C:1, 10, 100, 1000, 3000; gamma: 0.003, 0.01, 0.03, 0.1, 0.3, 1.) returning the highest R² score. Prediction accuracy was determined by overall evaluation and by pixel-by-pixel evaluation. The overall evaluation calculated an R² value by comparing all predicted GSVs with measured GSVs in the testing data set to assess the overall prediction accuracy of the model. Pixel-by-pixel evaluation calculated the R² value at each pixel to assess the prediction accuracy at different positions on LDPs. Accuracy percentages were calculated by counting pixels with an R² score larger than 0.90 (or between 0.79 and 0.85), and dividing by 720. The R² evaluation was performed with the metrics module on scikit-learn. The matplotlib module in Python was used for visualization of evaluation results and predicted images².

### Example IV

### Dark Area Calculation

In the machine-learning training process, we collected LDPs from a larger cuboid photobioreactor (19.6 cm in length×9.6 cm in width×20 cm in height) in order to get more information from a single image. However, the photobioreactor used for cultivation was a smaller photobioreactor 10 cm long and 5 cm wide. To adapt the pre-trained models to the smaller photobioreactor, we selected the 10 left-most columns (column 0-9) and 10 right-most columns (column 30-39) in the 9 rows (row 0-8) closest to the light source in the LDP of the larger photobioreactor to represent the LDP of the smaller photobioreactor. Thus, LDPs in small photobioreactors were represented by images with 180 (20×9) pixels. For dark area calculation, grayscale values less than 25.5 ( 1/10 of the maximum grayscale value) were counted (n) and normalized as percentages of LDP pixels (n/180×100%). The dark area with double light sources was estimated with the following equation (1), assuming no interference between light from two sources:

$${A}_{2} = \{ \begin{matrix}( 1 - ( 1 - {A}_{1} ) \times 2 ) \times 100 \% & if {A}_{1} > 50 \% ; \\ 0 & if {A}_{1} < 50 \% ;\end{matrix} \qquad ( 1 )$$

Where A₁ and A₂ refer to dark areas with one and two light sources at given light intensities, respectively.

### Example V

### Growth Curve Fitting, Growth Rate Calculation, and Biomass Productivity Prediction

To generate a growth curve, we collected cell concentration under given light intensities at different time points by measuring OD₇₃₀. Variables were normalized by 10 subtracting their average and dividing by their standard deviation. The logistic curve was defined as the equation (2):

$$f ( x ) = \frac{a}{( 1 + {e}^{- c ( x - d )} )} + b \qquad ( 2 )$$

Where x represents the variable here, representing time and a, b, c, d are parameters that determine the shape of the growth curve. The fitting and prediction were processed by the Optimize module in the SciPy library in Python³. Growth rates at specific time points were estimated by the slope of the corresponding curve at that point.

### Example VI

### Growth Rate Prediction Model (GRM) Training and Evaluation

The GRM was trained to predict cyanobacterial growth rates based on the LDPs (FIG. 7B). In order to collect training data, we cultivated cyanobacteria under different light intensities (107, 178, 267, 357, 570, 714 μmol m⁻² s⁻¹) in the smaller PBR. The concentration of the cyanobacteria was monitored and fitted with sigmoid curves for growth rate calculations mentioned above. Vectors extracted from the first 9 rows in the middle column of the LDP were used as features, and the corresponding (at the same time points) calculated growth rates were set as labels in the training. The features were normalized by subtracting their averages and dividing by their standard deviations. The random forest algorithm was used for the GRM model and the performance of the model was evaluated by calculating an R² value between the predicted and measured growth rates in the reserved testing set (20% of the training samples). The GRM was adapted to predict growth rates under a double-light condition based on the assumption that there are no interferences between light from two sources. In this way, vectors extracted from the first 5 rows of the middle columns of LDPs were used as features for the GRM training.

Similar to the GRM for PBR, we grew several batches of cyanobacteria in a pond system to acquire the growth data for pond GRM training. The growth data were then fitted with sigmoid curves for growth rate calculation. The normalized 208-length vectors predicted from the pond LDPM and the calculated growth rates were set as features and labels, respectively, for the pond GRM training. The training and evaluation of the pond GRM were the same as the PBR GRM described above.

### Example VII

### Growth Simulation

Cyanobacterial growth simulation was performed as shown in FIG. 7A. An initial cell concentration and light program are required inputs and the simulation process contains a loop with four steps: 1) predict the LDP based on the initial cell concentration and initial light intensity with the LDPM; 2) predict the growth rate based on the LDP from step 1 with the GRM; 3) calculate the new cell concentration from the initial cell concentration and the predicted growth rate; 4) update the newly calculated cell concentration and current light intensity as inputs for the next round of LDP prediction. The light programs were specified in the main text. The initial cell concentration used for PBR growth simulation ranged from 0.2 to 4.8, with a 0.2 increment. The initial concentration used for pond growth simulation was set to 0.1, 0.4, 0.6, and 0.8. To ensure accurate growth simulation, the bubbling rate, temperature, surface area, and light conditions were tightly controlled in a way that no severe sedimentation happens during cultivation in PBRs, while these conditions were controlled to achieve sedimentation in collection vessel.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in microbiology, molecular biology, biochemistry, chemistry, biofuel, and bioplastics, or related fields are intended to be within the scope of the following claims.

### Example VIII

### Measurement of NP Capture by Cyanobacteria

Cyanobacteria at an OD₇₃₀ of 15 was used for MP removal tests. In order to have enough samples for downstream suspension and sediment analysis, capture experiments were set up for 200 nm PS particles with volumes of 20 ml. Specifically, appropriate volumes of 10% PS stocks (Sigma, 69057) were directly mixed with 20 ml cyanobacterial suspensions to final concentrations of 0.001%, 0.005%, 0.01%, 0.02%, and 0.05%. The mixtures were seated for 1 hour to allow sufficient sedimentation. In order to accommodate the measurements of WT samples, where little or no sedimentation occurred, the upper 15 ml of mixture of each sample was carefully removed and seen as suspensions, while the remaining 5 ml sample was estimated as sediments, no matter if there was sedimentation or not. The measurements for 500 nm and 800 nm PS capturing were performed at a volume of 6 ml. Similarly, appropriate volumes of 10% 500 nm (Sigma, 59769) and 800 nm PS stocks (Sigma, 65984) were added to 6 ml of cyanobacterial suspension to final concentrations of 0.02% and 0.1%. Suspension measurements were carried out to quantify the PS capture after 1 h sedimentation.

### Example IX

### Suspension Analysis for 200 nm PS Removal by Cyanobacteria

The turbidity analysis quantifies the presence of overall cyanobacterial cells and PS particles in suspensions. Due to the huge turbidity difference between samples (especially for L524 samples with different concentrations of PS particles), appropriate dilutions were made to different samples to ensure the consistency of measurements. The turbidity was measured with the SpectraMax iD5 (Molecular Devices) spectrometer by reading absorbance at 850 nm. On the other hand, the chlorophyll fluorescence was used to estimate concentrations of cyanobacteria in the suspensions.

Differential centrifugation was employed to separate cyanobacterial cells and polystyrene MPs, and to quantify PS particles remaining in the suspension. The centrifugation speeds were set to 800×g for samples containing 200 nm PS particles and 300×g for 500 and 800 nm PS MPs, and the centrifugation time was set to 3 min. Decent separations were achieved through the centrifugation procedures, legitimating the effectiveness and reliability of the protocol. Subsequently, the relative PS abundance in suspension was calculated by normalizing the readouts from control groups as 100%. Specifically, the group of WT-0.05% PS was normalized to 100% for the testing of 200 nm PS MPs and the WT-PS groups were set to 100% in the experiments of 500 nm and 800 nm MPs.

### Example X

### Electron Microscopy Imaging Analysis

Sediments collected from the aforementioned procedures were first lyophilized for 48 hours before being analyzed with the scanning electron microscope. The images were recorded on a Tescan FERA-3 Model GMH Focused Ion Beam Microscope at an accelerating voltage of 5 kV. The transmission electron microscopy (TEM) is used to observe interactions between cyanobacterial cells and PS particles. The cyanobacteria-PS mixtures were first incubated for 30 mins to allow thorough interacting, and then gently resuspend sediments before taking samples. Droplets containing 2 μl samples were added onto copper grids, followed by leaving them on for 5 min to allow cell and particle settling. The remaining liquids were removed with filter paper before negative staining with 1% uranyl acetate for 20 seconds. The grids were then air-dry for several hours and observed under JEOL 1200.

### Example XI

### Mechanical Property Tests for Bioplastic Composite Films

The films were cut into strip-like shape, with a dimension of about 1.5 mm in width and 3 cm in length. The precise width (W) and thickness (T) were measured using a Vernier caliper and a Spiral micrometer, respectively. The strips were glued on a paper board sample holders and then fixed on the grippers. The original length (L) of strips was measured using a vernier caliper. A 100 Series Modular Universal Test Machines equipped with a 2 N load cell (TestResources, MN, US) was used to measure mechanical properties. The applied force (F) and corresponding displacement (d) were monitored synchronously during the measurement. The cross area (A) was calculated using the equation of A=W×T. Stress-strain curves can be plotted after getting stress (σ) and strain (ε) using the equations of σ=F/A and ε=d/L, respectively. The ultimate tensile strength was the maximum stress before fracture and the modulus of elasticity (MOE) was obtained from the slope of the elastic deformation region in the stress-strain curve. Elongation (%) was calculated using the equation of d′/L×100, where d′ is the displacement at the fracture. The toughness was the integral area under the stress-strain curve calculated using the Origin software (OriginPro 2021, US).

1. A method, comprising:
a) providing;
i) a plurality of photosynthetic cyanobacterial cells comprising a transgene comprising a codon optimized plant limonene synthase gene; and
ii) a body of water comprising a plurality of plastic particles;
b) overexpressing said transgene to display at least one limonene moiety on the surface of said plurality of photosynthetic cyanobacterial cells;
c) capturing a portion of said plurality of plastic particles with said displayed at least one limonene moiety to create a limonene-plastic particle complex; and
d) sedimenting said limonene-plastic particle complex.

2. The method of claim 1, wherein said photosynthetic cyanobacterial cells are Synechococcus (S.) elongatus UTEX 2973 cells.

3. The method of claim 1, wherein said body of water is a body of wastewater.

4-6. (canceled)

7. The method of claim 1, wherein said plurality of plastic particles comprise at least one polymer selected from the group consisting of polyethylene, polyethylene terephthalate, polystyrene and carboxymethyl cellulose.

8. The method of claim 1, wherein said limonene-plastic particle complex is a bioplastic composite.

9. The method of claim 1, wherein said plurality of plastic particles are a plurality of plastic microparticles or a plurality of plastic nanoparticles.

10-11. (canceled)

12. The method of claim 1, wherein said codon optimized limonene synthase gene is a plant limonene synthase gene or a mint limonene synthase gene.

13. (canceled)

14. The method of claim 12, wherein said mint limonene synthase gene is a Mentha spicata limonene synthase gene.

15. (canceled)

16. A composition comprising a photosynthetic cyanobacteria comprising a transgene comprising a codon optimized limonene synthase gene.

17. The composition of claim 16, wherein said photosynthetic cyanobacteria overexpresses a limonene synthase protein.

18. The composition of claim 16, wherein said codon optimized limonene synthase gene is a plant limonene synthase gene or a mint limonene synthase gene.

19. (canceled)

20. The composition of claim 184, wherein said mint limonene synthase gene is a Mentha spicata limonene synthase gene.

21. The composition of claim 16, wherein said photosynthetic cyanobacteria is Synechococcus (S.) elongatus.

22. (canceled)

23. The composition of claim 17, wherein said overexpressed limonene synthase protein displays a limonene moiety on the surface of said photosynthetic cyanobacteria cell.

24. The composition of claim 16, wherein said transgene is operably linked to a constitutive promoter.

25. (canceled)

26. The composition of claim 16, wherein said composition further comprises a wastewater.

27. The composition of claim 26, wherein said wastewater comprises a plurality of plastic nanoparticles and/or a plurality of plastic microparticles.

28. The composition of claim 27, wherein said plurality of plastic nanoparticles and/or said plurality of plastic microparticles comprise 0.5% (w/v) of said wastewater.

29. (canceled)

30. The composition of claim 27, wherein said plurality of plastic nanoparticles and/or said plurality of plastic microparticles comprise at least one polymer selected from the group consisting of polyethylene, polyethylene terephthalate, polystyrene and carboxymethyl cellulose.

31. The composition of claim 27, wherein said plurality of plastic nanoparticles comprise an average diameter of approximately 200, 400 or 800 and said plurality of plastic microparticles comprise an average diameter of approximately 1, 2 or 5 micrometers.

32-59. (canceled)
